SEQUENCE LISTING (5 sequences)

SEQ ID NO: 1 - protein, 142 aa, synthetic construct
GLNDIFEAQKIEWHEGPTGTGESKCPLMVKVLDAVRGSPAINVAMHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 2 - protein, 147 aa, Homo sapiens
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 3 - protein, 127 aa, Homo sapiens
GPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 4 - protein, 127 aa, Homo sapiens
GPTGTGESKCPLMVKVLDAVRGSPAINVAMHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 5 - protein, 15 aa, synthetic construct
GLNDIFEAQKIEWHE

(19) United States
(12) Patent Application Publication
(10) Pub. No.: US 2023/0184788 A1
(43) Pub. Date: Jun. 15, 2023

(54) A SYNTHENTIC POLY-TRANSTHYRETIN AFFINITY TRAP FOR DETECTING AMYLOIDOGENIC TRANSTHYRETIN FORMS IN PLASMA SAMPLES OFTRANTHYRETIN AMYLOIDOSIS PATIENTS

(71) Applicant: Northwestern University, Evanston, IL (US)

(72) Inventor: Jing Jin, Evanston, IL (US)

(21) Appl. No.: 18/048,673

(22) Filed: Oct. 21, 2022

Related U.S. Application Data
(60) Provisional application No. 63/262,900, filed on Oct. 22, 2021.

(51) Int. Cl. G01N 33/68; G01N 33/58; G01N 33/543

(52) U.S. Cl. CPC G01N 33/6893; G01N 33/58; G01N 33/54366; G01N 2800/32; G01N 2800/347

(57) ABSTRACT

Disclosed herein are methods for detecting the presence of transthyretin monomers in a subject sample.

### CROSS-REFERENCE TO RELATED APPLICATIONS

The present application claims the benefit of U.S. Provisional Application No. 63/262,900, filed Oct. 22, 2021, which is incorporated by reference herein in its entirety.

### STATEMENT OF GOVERNMENTAL RIGHTS

Not applicable.

### SEQUENCE LISTING

A Sequence Listing accompanies this application and is submitted as an .xml file of the sequence listing in ST26 format named “702581_02237.xml” which is 6,006 bytes in size and was created on Oct. 21, 2022. The sequence listing is electronically submitted via Patent Center with the application and is incorporated herein by reference in its entirety.

### FIELD

The field of the invention relates to methods for detecting monomeric transthyretin (TTR). In particular, the field of the invention relates to methods for detecting monomeric TTR in a biological sample by adding a variant TTR that binds monomeric TTR and forms a detectable complex.

### BACKGROUND

Transthyretin amyloidosis (ATTR) is a chronic disease caused by the formation and accumulation of amyloid transthyretin (TTR) fibrils in organs. TTR is an abundant plasma protein produced in the liver. Aberrant TTR resulting from either genetic mutations or old age is prone to adopt an amyloid fold and form fibril deposits in virtually all tissues, causing cardiac and nervous system dysfunctions. Plasma TTR functions as a carrier protein in circulation and naturally exists as a tetramer. However, mutations or biochemical environments such as oxidation may cause disassembling of tetrameric TTR to form TTR monomers. The monomer is unstable and has a tendency to adopt amyloid fold and fibril formation. In light of TTR stabilizers entering the drug market and new treatment for ATTR, diagnostic methods for measuring blood TTR monomers are urgently needed. These diagnostic tools will not only evaluate the efficacy of TTR stabilization drugs but also give prognoses of ATTR severity: higher TTR monomer levels are associated with worse clinical outcomes. Methods for specifically detecting TTR monomers are being invented using monoclonal antibodies. However, the efficacy of the antibody method has not been confirmed in clinical tests.

### SUMMARY

Disclosed are methods and compositions for detecting transthyretin (TTR) monomers. Particularly disclosed are methods and compositions for detecting TTR monomers in a biological sample from a subject.

In some embodiments, the disclosed methods comprise: (a) contacting the sample with a TTR binding reagent for a sufficient time for the TTR binding reagent to bind to TTR monomers in the sample and form a complex comprising the TTR binding reagent and oligomerized TTR monomers, the TTR binding reagent comprising: (i) a variant TTR comprising a valine to methionine substitution at position 30 (TTRV30M); and (ii) an affinity moiety linked to the variant TTR; and (b) detecting the presence of the complex. In some embodiments, the affinity moiety is biotin. In some embodiments, the affinity moiety is linked to the N-terminus of the TTR binding reagent. In some embodiments, the TTR binding reagent has the sequence of, or is at least about 90% similar to that of SEQ ID NO: 1. In some embodiments, the method further comprises binding the complex to streptavidin. In some embodiments, the TTR binding reagent is linked to a solid support. In some embodiments, the solid support comprises a microplate or a bead. In some embodiments, detecting comprises contacting the complexes with a detection reagent. In some embodiments, the detection reagent is selected from the group comprising an antibody, an aptamer, a fluorescent reagent, a chemiluminescent reagent, and a colorimetric reagent. In some embodiments, the antibody or aptamer is linked to a detectable marker comprising a fluorescent molecule, a luminescent molecule, a radioisotope, and an enzyme capable of catalyzing a detectable chemical reaction.

### BRIEF DESCRIPTION OF THE DRAWINGS

FIG. 1. Biotin-TTR fusion and SA-induction model. (A) Crystal structure of tetrameric TTR (PDB 5H0V) (58). The 2 sets of antiparallel β-strands that form the β-sheets of DAGH and CBEF were marked in 1 of the subunits (upper-right). Biotin moieties (represented by spheres) were added to the N-termini of all TTR subunits. (B) Schematics of site-specific biotinylation by BirA and subsequent multimeric induction by SA. Full-length TTR variants (asterisks) of WT, V30M, and V122I were each fused with an N-terminus AviTag, which was biotinylated. In the presence of SA (lower left: follow arrow), 4 biotin-TTR monomers formed SA-TTR tetramers. In the absence of SA (right panel: follow arrow), soluble TTR formed a mixture of monomer (M), dimer (D), and tetramer (T), in which the broken circle represents the natural tetrameric fold of TTR. Following SA induction of these mixed TTR forms (Bottom right: follow arrow), larger TTR complexes were formed, jointed by monomer, dimer, and tetrameric TTRs (in box). (C) WT, V122I, and V30M TTRs in the presence or absence of SA were resolved by reducing SDS PAGE, and subsequently stained with Coomassie blue. Without induction by SA, all variants existed predominantly as SDS-resistant dimers with the presence of less abundant monomers. Following SA induction, TTR formed larger complexes of greater than 600 kDa. (D) Biotinylated TTRWT in 1 M urea pH 7.6 solution formed 3 distinct complexes of M, D, and T as revealed by SEC (left panel). Following induction by SA (right panel), TTR in multimeric complexes with SA gained molecular size (the red line for after induction compared with the solid black line for before induction; SA alone: dotted line). The results of V30M and V122I TTRs with SA induction are in FIG. 8.

FIG. 2. TTR monomer vs. dimer vs. tetramer distribution among WT, V30M, and V122I variants at pH 7.6 in 1 M urea. Recombinant TTR proteins were subjected to either PBS at pH 7.4 (top panels) or 1 M urea condition at pH 7.6 (bottom panels). SEC analyses showed all variants stayed predominantly as tetramers in PBS, as expected. However, TTR separated into monomer (M), dimer (D), and tetrameric (T) forms under 1 M urea condition. In addition, the individual variants of TTRWT, TTRV30M, and TTRV122I showed different distributions of their monomeric, dimeric, and tetrameric contents. All experiments were repeated 3 times.

FIG. 3. Dynamics in oligomeric transformation among TTR variants of WT, V30M, and V122I. (A) Freshly thawed TTR proteins in 1 M urea pH 7.6 solution were incubated at room temperature for up to 7 days. In between, aliquots were taken from the reaction at indicated time points of 0, 1, 3, 6, and 24 hours, and 3 and 7 days. Left panels compared SEC profiles between 0 and 7 days in terms of monomeric (M), dimeric (D), tetrameric (T) and oligomeric (O) contents. The right panel bar graphs showed the percentage distribution among M, D, R, and O contents as calculated from AUC of the respective peaks over the 7 day period. (B) Treatment of TTR solutions with 100 µM H2O2 further elevated the levels of O (compare A with B), particularly in V30M. (C) Treatment of TTRV30M with 5 mM DTT greatly increased tetramers (compare B with C). Meanwhile, the D contents completely disappeared. (D) Monomeric, dimeric, and tetrameric contents of TTRV30M were separated and collected by SEC. The fractions with the presence or absence of TCEP were resolved by SDS PAGE. TTR bands were visualized by IB using anti-TTR antibody. All experiments were repeated 3 times.

FIG. 4. Rapid coaggregation of TTRWT monomers by artificially induced SA-TTR complexes. (A) TTRWT was incubated in the presence or absence of SA-induced TTRV30M seeds (right and left panels, respectively; preparation of induced TTRV30M seeds is shown in FIG. 8) in 10:1 w/w ratio for a total of 180 minutes. Aliquots were taken from the reaction at indicated time points and subsequently analyzed by SEC. The seeds-alone sample was separately analyzed by SEC. Monomeric (M) dimeric (D), tetrameric (T), and oligomeric (O) contents are indicated. Insets below show the M peaks in the time series. The arrow shows the range of O increases on top of the seed amount. Additional results of TTRWT, TTRV30M, and TTR- V122I with the seeds are in FIGS. 10 and 11. (B) Monomeric content of TTRWT before induction was purified by SEC and then subjected to conditions with or without the TTRV30M seeds (top-left and bottom-left panels, respectively). Aliquots taken from 0, 1 (immediately after the spiking-in of seeds), 10, and 60 minutes were analyzed by SEC. There was the contrasting difference between the presence or absence of the seeds. Without the seeds, there was a gradual shift of monomer contents to newly formed dimers and tetramers. With the seeds, there was a rapid increase of high molecular weight of TTR oligomers (arrow indicates the change in levels). Within minutes, the multimeric seeds depleted most monomers in forming high molecular weight protein complexes. In contrast to TTRWT monomers, dimeric and tetrameric fractions did not react to seed-induced aggregation. All experiments were repeated 3 times.

FIG. 5. Construction of synthetic poly-TTRV30M-based ELISA to detect misfolded TTR contents in serum. Given that SA-clustered synthetic TTR polymer can specifically coalesce monomeric forms of TTR in solution as analyzed by SEC (FIG. 4), here we immobilized SA-induced poly-TTRV30M seeds on ELISA plates. (A and B) The coated wells were incubated with various concentrations of either WT (A) TTRWT with TMT label for detection using anti-TMT antibody; details in Methods) or V30M (B) TTRV30M with TMT label) recombinant proteins. The control wells contained unin- duced TTRV30M seeds, which did not present high avidity compared with SA-clustered TTRV30M seeds. As expected, wells coated with the clustered TTRV30M seeds bound both TTRWT and TTRV30M. Similarly, for testing whether this ELISA methodology using clustered TTRV30M can detect unstable and potentially amyloidogenic TTR species in blood samples, the detection kit was applied to human serum. Whole serum proteins were labeled with TMT, and following different dilutions, the samples were incubated with the ELISA plate. Serum proteins captured by clustered TTRV30M on the plate were detected by anti-TMT antibody. (C and D) As controls, purified recombinant TTRWT (C) or TTRV30M (D) (also labeled with TMT) as the spike-in was added to the serum samples at indicated concentrations. The results showed the methodology capable of detecting unstable TTR contents in serum.

FIG. 6. SA-TTR multimer formed renal deposits in mice. (A) A total of 25 mice (n = 5/group) received 7 i.v. doses of buffer, uninduced TTRWT, or SA-induced TTRWT, TTRV30M, or TTRV122I at 15 mg/kg BW per dose for 7 consecutive days (arrows). The mice were harvested (arrowhead) 3 hours after the last injection on day 7. Kidney, liver, and heart specimens were stained with anti-TTR antibody by IF. (B) IF images of the kidney sections showed no TTR signal from the buffer and only a trace amount of uninduced TTR in some, but not all, glomeruli (top panels). In contrast, all SA-induced TTR variants in their multimeric forms showed prominent TTR signals as puncta of varying sizes exclusively in the glomerulus (arrowheads and circled areas). All glomeruli were stained positive, whereas renal tubules only showed background-level signals with no TTR puncta (bottom panels). (C) Inset of SA-induced V30M image from bottom center panel in the boxed area of A. (Images from induced TTRWT and TTRV122I injections are in FIG. 12). CD31 marked glomerular capillaries. Cross sections of the capillary loops are pointed by arrowheads (left panel of CD31 staining). Strong SA-TTR deposition was observed with aggregates varying in size and shape (middle panel). Composite image of the glomerulus showed TTR deposits were not in the capillary lumen (arrowheads). Instead, the deposits were located to the glomerulus mesangial areas. (D) The liver of SA-V30M-injected mice showed scattered TTR puncta that also appeared to be outside of vascular lumina (marked with CD31). Scale bar: 30 µm.

FIG. 7. SA-TTR multimer formed cardiac deposits following i.v. injection in mice. (A) Mice (n = 5/group) from the same injection series as in FIG. 6 were collected for histologic analysis of the heart. (B) IF staining of TTR was performed to the heart specimens. Representative images are shown for each group. SA-induced groups showed positive staining of TTR deposits (black dots: arrowheads pointed). Additional images of V30M deposition are in FIG. 13. (C and D) Mice injected with SA-induced TTRV30M showed cardiac TTR deposits. The deposits partially overlapped with WGA and CD31 staining signals of cardiac sarcolemma in C and vasculature in D. Scale bar: 50 µm.

FIG. 8. Biotinylated TTR variants of V122I and V30M were induced for multimerization by streptavidin (SA). Uninduced TTRs had mono- (M), di- (D) and tetrameric (T) contents as shown by SEC (black lines). Following induction with SA (as shown in FIG. 1D), the combined SA-TTR multimeric complexes had increased molecular sizes of ~670 kDa (red lines). SA alone was represented by the dotted lines.

FIG. 9. TTR variants of WT, V30M and V122I were prone to aggregate under low pH. Recombinant TTR proteins in either 1 M urea or PBS solution were subjected to pH 4.4 acetate buffer. Solution turbidity was monitored over a period of 96 hours.

FIG. 10. Dynamic changes of oligomer and monomer contents of TTR variants. Detailed view of FIG. 3A for within a 24 hour period. Changes in oligomer and monomer contents are shown in insets (left and right panels, respectively).

FIG. 11. Derived from FIG. 4A with insets showing details Left and right panels in A and B show selected regions of SEC graph of oligomers (O, left panels) and dimer (D and monomers (M) (right panels). In B with the spike-in of V30M seeds (10% ratio), there was a gradual accumulation of oligomeric contents (Left panel, follow arrow).

FIG. 12. Renal deposition of streptavidin-induced TTRWT and TTRV122I complexes. Similar to the experiment shown in FIG. 5, mice were intravenously injected daily with SA-induced TTRWT, TTRV122I, or uninduced TTRWT as control for seven consecutive days. Kidneys were harvested 3 hours after the last injection and specimens were stained with anti-TTR antibody with anti-CD31 as counterstain. Both SA-induced proteins showed prominent glomerular (circles and arrowheads) deposition, similar to the results from SA-V30M injection (FIG. 5). In contrast, uninduced TTRWT did not form deposits in the kidney.

FIG. 13. Transmission electron micrographs of glomerular TTR deposits from injection. Injection of streptavidin (SA: control) did not form kidney deposits in the glomerulus (upper left; MC: mesangial cell; fp: foot processes; RBC: red blood cell). Meanwhile, i.v. injection of SA-induced TTR oligomer complexes had led to deposition of electron-dense materials in the glomerulus (upper right; box 1: pointed by arrow). Lower panels show the electron-dense deposit at higher magnifications.

FIG. 14. Cardiac deposits of SA-induced TTRV30M (an overview of FIG. 6). Mouse injected with SA-induced TTRV30M with heart specimen stained with anti-TTR antibody. Upper left: a broad view of anti-TTR immunofluorescence image of the heart. Insets a-e show selected areas (red boxes) at higher magnification of TTR deposits in myocardium.

FIG. 15. Streptavidin-induced TTR deposits in the heart and in the kidney completely disappeared after 4 weeks (as compared to FIG. 6 and FIG. 7).Mice (n=3 in each group) were injected daily with either SA control, uninduced recombinant TTRV30M, or SA-induced recombinant TTRV30M oligomers for 7 consecutive days. Four weeks after the last injection, the hearts and the kidneys were collected for triple staining with anti-TTR, WGA and DAPI for the heart specimens (A), and with anti-TTR, anti-CD31 and DAPI for the kidney (B). Representative immunofluorescence images are shown. No TTR deposit signals (in green) were observed in the intermyofibrillar space of the heart (marked by WGA) and the glomerulus of the kidney (follow CD31 staining of the glomerular capillary). Scale bar: 25 µm

FIG. 16. A modest increase of thioflavin T (ThT) signals with recombinant TTR following using with synthetic oligomeric TTRV30M. In a 10:1 (w/w) ratio, 1 mg/ml of either TTRWT or TTRV30M (mixtures of monomers and tetramers) were added to microtiter plates with the presence or absence of 0.1 mg/ml synthetic TTRV30M oligomeric seeds from streptavidin induction. With 25 µM ThT in the reactions under shaking, ThT fluorescence signals at 450 nm excitation/485 nm emission were read at indicated time points. There was a modest increase in the signals following the addition of synthetic poly-TTRV30M seeds, occurring more rapidly within the first 0.25 hour.

### DETAILED DESCRIPTION

Recently, transthyretin (TTR) stabilizers, e.g., tafamidis, have entered the market for use in the treatment of transthyretin cardiac amyloidosis (TTR-CA). TTR stabilizers function by preventing the destabilization of TTR protein tetramer due to either inherited mutations (ATTRm) or the aging process in wild-type disease (ATTRwt). Destabilization of TTR tetramers promotes its dissociation into monomers, which misassemble into amyloid fibrils and deposit in tissues and organs. In the heart, deposition of amyloid fibrils in the extracellular space leads to diastolic dysfunction, progressing to heart failure with a restrictive physiology and eventual death. More information regarding the use and clinical efficacy of TTR stabilizers can be found in Rosenblum, H. et al. “TTR Stabilizers are Associated with Improved Survival in Patients with Transthyretin Cardiac Amyloidosis” Circulation: Heart Failure. 2018 Apr; 11(4) which is incorporated by reference herein in its entirety. Thus, by way of example but not by way of limitation, methods to accurately measure the TTR monomers in patient samples are urgently needed. Such diagnostic tools measure both the efficacy of TTR stabilization, but also provide prognostic data regarding the severity of disease, with the level of TTR monomers in a subject being correlated with a worse prognosis.

Amyloidosis involves stepwise growth of fibrils assembled from soluble precursor proteins. There is a focus in the field on understanding the folding energetics that drive self-assembly. Mutations of plasma carrier protein transthyretin (TTR) are associated with hereditary transthyretin amyloidosis (ATTR). TTR naturally folds into a stable tetramer for its function, whereas conditions and mutations that foster aberrant monomer formation facilitate TTR oligomeric aggregation and subsequent fibril extension. The inventors investigated the dynamics of early assembly of oligomers by wild-type TTR, in comparison to its TTRV30M and TTRV122I disease mutants. The inventors selected buffer conditions for monitoring time-dependent redistribution among monomer, dimer, tetramer, and oligomer contents in the presence and absence of multimeric TTR seeds. The seeds were artificially constructed recombinant multimers that contained 20- 40 TTR subunits via engineered biotin-streptavidin interactions. As expected, these multimer seeds rapidly nucleated TTR monomers and dimers into larger complexes, while having no effect on tetramers. In vivo, streptavidin-induced multimers formed ATTR-like deposits in the heart and the kidney following i.v. injection in mice. While all three variants prominently deposited in the kidney glomerulus, only TTRV30M resulted in extensive deposition in the heart. The cardiac TTR deposits varied in size and shape and were localized to the intermyofibrillar space along the capillaries. These results are consistent with the notion of monomeric TTR to engage high-avidity interactions with tissue amyloids. Critically, the inventors demonstrated that the TTRV30M multimers selectively bind to the monomeric TTR, i.e., the most amyloidogenic form of TTR.

Transthyretin amyloidosis (ATTR) is a systemic disease caused by tissue deposition of TTR fibrils. TTR is an abundant plasma protein mainly produced by the liver. The best characterized function of TTR is as a thyroxine-4 hormone and retinol-binding protein transporter. ATTR deposits of wild-type TTR are associated with acquired/senile amyloidosis that mainly affects the heart. There is a large number of genetic variants of TTR that cause autosomal-dominant ATTR with amyloid deposits in virtually every tissue of the body, most prominently affecting the nervous system, the heart, the eye, the gastrointestinal system, and the vasculature of the brain. Depending on the individual amino acid substitutions, the clinical course of hATTR varies in terms of onset age, and specific tissue and organ involvements. For example, V30M (also termed TTRV30M) is the most common hATTR variant that causes TTR familial amyloid polyneuropathy (ATTR-FAP). TTRV30M is prevalent among the Caucasian population, particularly in Portugal and Sweden (1). Meanwhile, V122I (TTRV122I) is another common genetic variant of hATTR, and its carriers have elevated risks of heart failure due to cardiomyopathy (ATTR-CM) among individuals of African or Hispanic ancestry (2, 3). More than 140 TTR variants have been identified with different but overlapping spectra of onset age, clinical manifestations, and risks (4).

TTR protein in its native fold exists as a homo-tetramer with two funnel-shaped thyroxine-binding sites at its dimer-dimer interface (5). Drugs that stabilize TTR tetramers have been developed for treatment of ATTR (6, 7). Ex vivo studies demonstrated that most known hATTR mutants, including V30M and V122I, have a higher tendency than their wild-type counterpart to misfold and form unstable alternatives of monomers and dimers (PMID: 11752443, PMID: 27589730). The structure of TTR monomer contains eight β-strands, forming two four-stranded anti-parallel sheets, termed DAGH and CBEF. The DAGH sheet has the tendency of conformational changes in forming amyloid fibril (8, 9). Although the exact role of dimeric TTR remains unclear as to whether it is an intermediate of tetrameric to monomeric, or monomer to oligomer, transformation, its abundance is high in TTRV30M genetic background. From a structural perspective, it was noted that TTRV30M dimer is connected via an aberrant disulfide bridge facilitated by the steric conformation of the mutant (10, 11). Extensive studies investigated the structural basis for TTR destabilization in subsequent formation of amyloid fibrils (12, 13). It seems that individual pathogenic variants each has distinct characteristics to render amyloidogenic potentials. It should also be noted that, wild-type TTR, either in its full length or a cleaved form (14), is present not only in nonhereditary ATTR amyloid fibrils, but also in familial deposits either with the accompanying TTR variant (15, 16) or onto existing mutant seeds even after liver transplantation (17-19). In domino liver transplantation using explanted livers from hATTR-FAP donors, organ recipients have the risk of developing systemic amyloidosis (20, 21). Factors that accelerate amyloid fibrillogenesis are being investigated in the context of liver transplantation, including older age of domino liver recipients, as well as the potential of undetectable amyloid nuclei to cause disease transmission in recipients (22). These observations illustrate the importance of amyloid self-seeding, or cross-seeding between wild-type and disease variants, in accelerating the growth of TTR fibrils (22, 23).

TTR fibrils extracted from tissues are large complexes with thousands of protein subunits, which also contain other amyloid and non-fibrillar constituents including serum amyloid P component and glysosaminoglycans that present in all types of amyloid. As unfolded monomeric and dimeric TTRs are prone to form the initial oligomers, these small TTR oligomers are cytotoxic once seeded in tissues with the tendency to nucleate misfolded TTR forms from blood (24-29). Amyloid seeding has been broadly studied in many disease types of amyloidosis beyond ATTR (30-32). With regard to TTR, compelling evidence for its amyloid seeding effects came from the observation of hATTRV30M polyneuropathy patients who had undergone liver transplantation and subsequently developed cardiac deposition of wild- type TTR (18, 21). Recently, Eisenberg and colleagues provided direct evidence on amyloid fibrils extracted from heart tissues of ATTR patients in promoting nucleation of wild-type and monomeric TTR ex vivo (22).

Considerable amount of work has been on determining the thermodynamic forces that drive self- assembly of amyloid fibrils as well as the earlier aggregation of misfolded TTR to form intermediates and oligomers (24, 28, 33-36). To further investigate the interactions among TTR assemblies of monomer, dimer, tetramer, and oligomer, the inventors devised a recombinant fusion tag on the protein in order to artificially induce TTR polymerization via the tag. By adding this induced TTR polymer as nucleation seed to a mixture of TTR monomer, dimer and tetramer, the inventors examined the dynamic changes of these TTR forms in response to the seed.

Transthyretin amyloidosis is a chronic debilitating disease with systemic deposition of transthyretin fibrils in tissues, affecting the heart, the nervous system, the kidney, and vasculature. Transthyretin is an abundant plasma protein that has intrinsic tendency of forming self-assembled fibrils in a β-sheet helix structure. Ex vivo and in vivo data showed that many amyloids, including transthyretin, extend fibrils through nucleating their soluble precursors. The inventors devised an artificial transthyretin seeding model using an inducible transthyretin multimer, which showed robust nucleation of monomeric transthyretin proteins with the seed. Intravenous injection of the induced multimers in mice formed extensive cardiac deposits of transthyretin, suggesting that high avidity presented by the seeding subunits is sufficient to coalesce soluble transthyretin in self-aggregation.

The present invention is described herein using several definitions, as set forth below and throughout the application.

### Definitions

The disclosed subject matter may be further described using definitions and terminology as follows. The definitions and terminology used herein are for the purpose of describing particular embodiments only and are not intended to be limiting.

### Methods

In one aspect of the current disclosure, methods for detecting transthyretin (TTR) monomers in a biological sample from a subject are provided. In some embodiments, the methods comprise: (a) contacting the sample with a TTR binding reagent for a sufficient time for the TTR binding reagent to bind to TTR monomers in the sample and form a complex comprising the TTR binding reagent and oligomerized TTR monomers, the TTR binding reagent comprising: (i) a variant TTR comprising a valine to methionine substitution at position 30 (TTRV30M); and (ii) an affinity moiety linked to the variant TTR; and (b) detecting the presence of the complex.

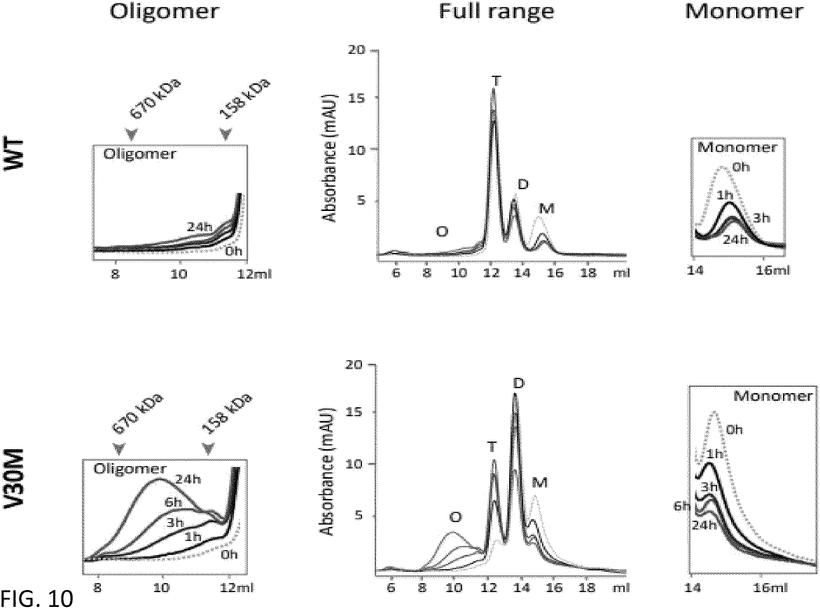

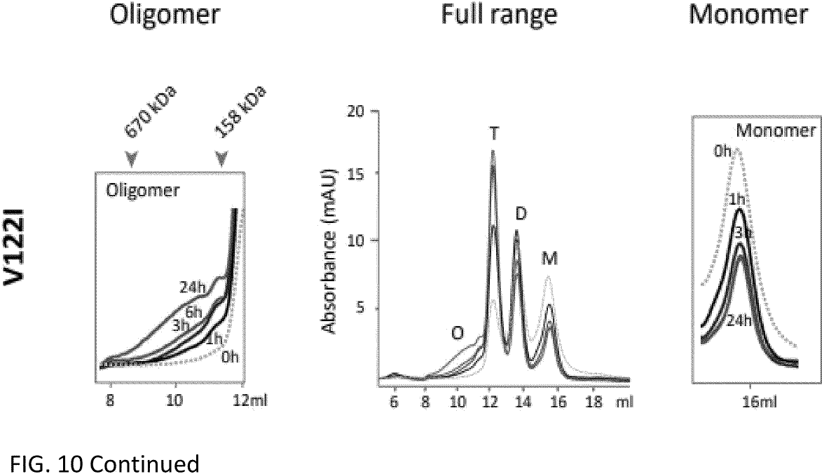

Disclosed herein is the unique property of multimerized TTRV30M to bind and act as an “affinity trap” for monomeric TTR. As used herein, “affinity trap” is a molecule that specifically and strongly binds to another target molecule. In some embodiments, the TTR binding reagent is an affinity trap for monomeric TTR. See, for example, FIG. 10 which demonstrates that addition of TTR binding reagent or “seeds” rapidly binds the TTR monomers “M”, exemplified by the increase in the height of the peak “O” or oligomers, and decrease in the height of the peak “M” after addition of TTR binding reagent.

As used herein, “transthyretin” or “TTR” refers to a transport protein present in the serum and cerebrospinal fluid that carries the thyroid hormone thyroxine (T₄) and retinol-binding protein bound to retinol. The liver secretes transthyretin into the blood, and the choroid plexus secretes TTR into the cerebrospinal fluid. Human transthyretin precursor protein has the sequence: MASHRLLLLC LAGLVFVSEA GPTGTGESKC PLMVKVLDAV RGSPAINVAV HVFRKAADDT WEPFASGKTS ESGELHGLTT EEEFVEGIYK VEIDTKSYWK ALGISPFHEH AEVVFTANDS GPRRYTIAAL LSPYSYSTTA VVTNPKE (SEQ ID NO: 2).

Mature TTR has the sequence: GPTGTGESKC PLMVKVLDAV RGSPAINVAV HVFRKAADDT WEPFASGKTS ESGELHGLTT EEEFVEGIYK VEIDTKSYWK ALGISPFHEH AEVVFTANDS GPRRYTIAAL LSPYSYSTTA VVTNPKE (SEQ ID NO: 3). In some embodiments, TTR is a variant with a V30M, i.e., a valine to methionine substitution at position 30 of the mature form of TTR with the sequence: GPTGTGESKC PLMVKVLDAV RGSPAINVAM HVFRKAADDT WEPFASGKTS ESGELHGLTT EEEFVEGIYK VEIDTKSYWK ALGISPFHEH AEVVFTANDS GPRRYTIAAL LSPYSYSTTA VVTNPKE (SEQ ID NO: 4). In some embodiments, an AviTag sequence is added to the N-terminus of TTR. In some embodiments, AviTag-TTR has the sequence: GLNDIFEAQKIEWHE GPTGTGESKC PLMVKVLDAV RGSPAINVAM HVFRKAADDT WEPFASGKTS ESGELHGLTT EEEFVEGIYK VEIDTKSYWK ALGISPFHEH AEVVFTANDS GPRRYTIAAL LSPYSYSTTA VVTNPKE (SEQ ID NO: 1). The AviTag facilitates directed biotinylation of the N-terminus by BirA enzyme. More information regarding the use of AviTag can be found in Beckett D. et al. “A minimal peptide substrate in biotin holoenzyme synthetase-catalyzed biotinylation” Protein Science, 1999. 8:921-929 which is incorporated by reference herein in its entirety. In some embodiments, additional tags are added to SEQ ID NOs: 1-4 including, for example, a 6xHis tag, or other such synthetic or naturally-derived tags that are well known in the art, e.g. flag, or glutathione S-transferase tags.

As used herein, “monomer” refers to a single unit of a larger polymer, such as a protein multimer. As used herein, “multimer” refers to a protein comprising multiple monomers. In some embodiments, a single unit of TTR, or modified TTR such as, for example, the protein with SEQ ID NO: 1 is referred to as a monomer. In some embodiments, multiple chemically-linked monomers of TTR or the protein with sequence of SEQ ID NO: 1 are referred to as multimers.

As used herein, “biological sample” refers to a sample derived from a biological organism, i.e., a subject. In some embodiments, a biological sample is, for example, plasma from a human (Homo sapiens). In some embodiments, a biological sample is a sample, e.g., plasma, cerebrospinal fluid, blood, tears, urine, feces, or a tissue biopsy, derived from a human suspected of or known to be suffering from amyloidosis including familial amyloid polyneuropathy (FAP). However, it will be understood by one of skill in the art that additional or alternative samples may be used in the practice of the disclosed methods. A factor for selection of suitable samples for use in the disclosed methods is the suspected presence, or confirmed presence, of TTR monomer in the type of sample used in the method. In some embodiments, the subject is known to have a TTRV30M, or TTRV122I mutation.

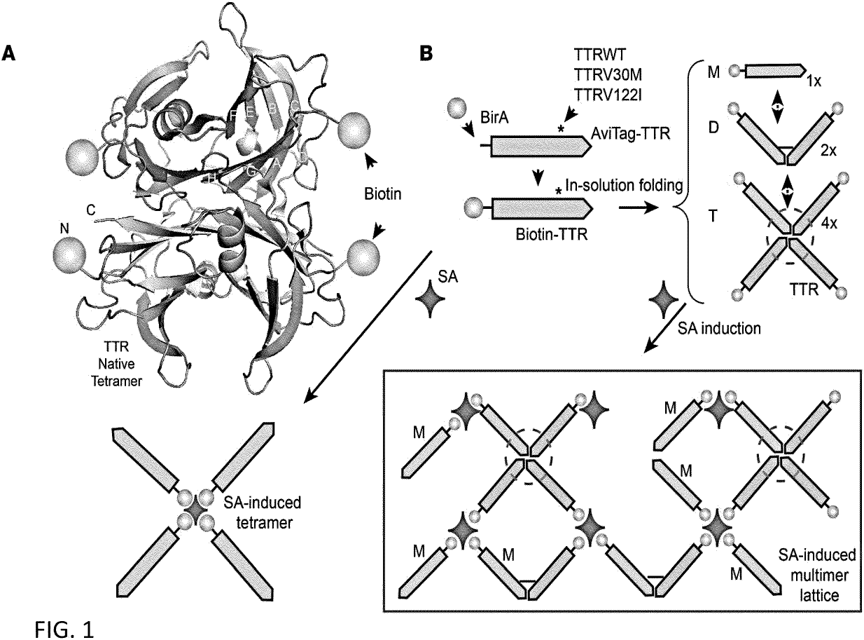

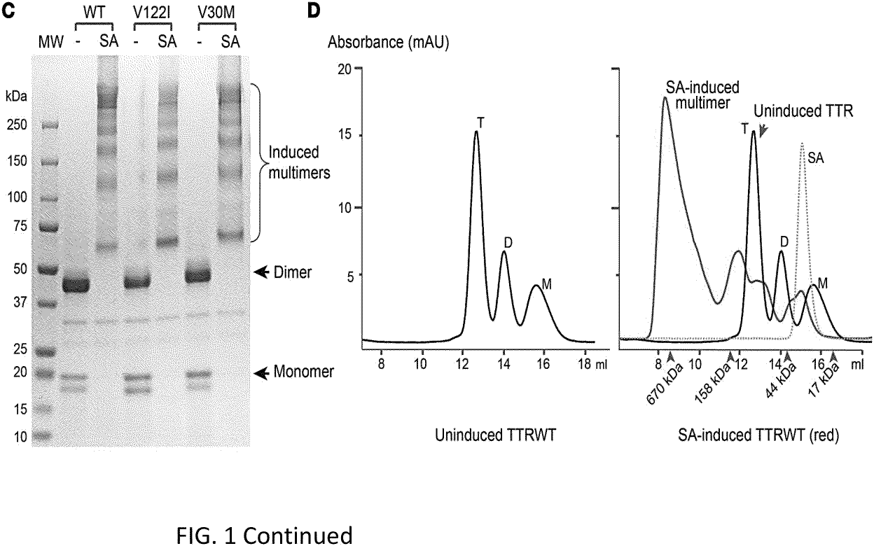

As used herein, “TTR binding reagent” refers to multimerized monomers of TTR V30M. In some embodiments, TTR binding reagents are comprised of monomers with a sequence at least 90% similar to SEQ ID NO: 1. In some embodiments, monomers are selectively biotinylated on the N-terminus. Biotinylated monomers are then multimerized in the presence of streptavidin. Without being bound by any theory or mechanism, it is believed that the binding of the biotinylated N-termini of the TTRV30M monomers to streptavidin leaves the C-termini free to associate with other TTRV30 molecules creating a multimerized network of TTRV30M proteins that are able to selectively bind to monomeric TTR in a sample (FIG. 1).

As used herein, “complex” refers to two or more associated biological macromolecules.

As used herein, “detecting” refers to the process of confirming the presence, either qualitatively or quantitatively, of a molecule in a sample. In some embodiments, detecting is performed by the human eye. In some embodiments, detecting is performed automatically by a device and does not require the use of human mental faculties.

As used herein, “detection reagent” refers to any reagent that functions in conjunction with the TTR binding reagent such that the combination of the TTR binding reagent and the detection reagent allows for detection of TTR binding reagent-TTR monomer complexes in the sample. In some embodiments, detection reagents comprise an antibody, an aptamer, a fluorescent reagent, a chemiluminescent reagent, and a colorimetric reagent. In some embodiments, the antibody or aptamer is linked to a detectable marker comprising a fluorescent molecule, a luminescent molecule, a radioisotope, and an enzyme capable of catalyzing a detectable chemical reaction.

As used herein, “affinity moiety” refers to a chemical constituent, often attached to a molecule of interest that can be specifically recognized and bound by a “binding reagent” with high affinity, and with high binding strength suitable to allow near covalent-strength binding of the molecule of interest to which the affinity moiety is attached. By way of example, but not by way of limitation, exemplary affinity moiety and binding reagent pairs include biotin and streptavidin, digoxigenin and anti-digoxigenin antibodies, antibody-antigen pairs, and covalent click chemistry.

As used herein, “support” refers to any material having a surface onto which one or more fluids may be deposited. The support may be constructed in any of a number of forms such as wafers, slides, well plates, membranes, for example. In addition, the support may be porous or nonporous as may be required for deposition of a particular fluid. Suitable support materials include, but are not limited to, those supports that are typically used for solid phase chemical synthesis, e.g., polymeric materials (e.g., polystyrene, polyvinyl acetate, polyvinyl chloride, polyvinyl pyrrolidone, polyacrylonitrile, polyacrylamide, polymethyl methacrylate, polytetrafluoroethylene, polyethylene, polypropylene, polyvinylidene fluoride, polycarbonate, divinylbenzene styrene-based polymers), agarose (e.g., Sepharose®), dextran (e.g., Sephadex®), cellulosic polymers and other polysaccharides, silica and silica-based materials, glass, and functionalized glasses, ceramics, and such substrates treated with surface coatings, e.g., with microporous polymers (particularly cellulosic polymers such as nitrocellulose), microporous metallic compounds (particularly microporous aluminum), antibody-binding proteins (available from Pierce Chemical Co., Rockford Ill.), bisphenol A polycarbonate, or the like. In some cases, array supports are porous surfaces or gels such as methacrylates, acrylamides, sugar polymers, cellulose, silicates, and other fibrous or stranded polymers. As used herein, “solid support” refers to a solid-phase “support” as defined above.

The term “subject” may be used interchangeably with the terms “individual” and “patient” and includes human and non-human subjects. In some embodiments, subjects are human or Homo sapiens.

### Kits

In another aspect of the current disclosure, kits are provided. In some embodiments, the kits comprise a TTR binding reagent comprising: (i) a variant TTR comprising a valine to methionine substitution at position 30 (TTRV30M); and (ii) an affinity moiety molecule linked to the variant TTR.

The disclosed kits may be used to detect free TTR monomer, e.g., misfolded TTR monomer in a sample from a subject. In some embodiments, the TTR binding reagent binds to TTR monomer in a TTR binding buffer. As used herein, “TTR binding buffer” refers to a suitable buffer for example tris buffered saline (TBS) or tris buffered saline plus tween (TBST). Suitable buffers for allowing protein-protein interactions to occur are known in the art.

A DNA sequence that “encodes” a particular RNA is a DNA nucleic acid sequence that is transcribed into RNA. A DNA polynucleotide may encode an RNA (mRNA) that is translated into protein, or a DNA polynucleotide may encode an RNA that is not translated into protein (e.g., tRNA, rRNA, or a guide RNA; also called “non-coding” RNA or “ncRNA”). A “protein coding sequence” or a sequence that encodes a particular protein or polypeptide, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from prokaryotic or eukaryotic mRNA, genomic DNA sequences from prokaryotic or eukaryotic DNA, and synthetic nucleic acids. A transcription termination sequence will usually be located 3′ to the coding sequence.

The phrases “% sequence identity,” “percent identity,” or “% identity” refer to the percentage of amino acid residue matches between at least two amino acid sequences aligned using a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

The terms “protein,” “peptide,” and “polypeptide” are used interchangeably herein and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. A protein may comprise different domains, for example, a nucleic acid binding domain and a nucleic acid cleavage domain. In some embodiments, a protein comprises a proteinaceous part, e.g., an amino acid sequence constituting a nucleic acid binding domain.

Nucleic acids, proteins, and/or other compositions described herein may be purified. As used herein, “purified” means separate from the majority of other compounds or entities, and encompasses partially purified or substantially purified. Purity may be denoted by a weight by weight measure and may be determined using a variety of analytical techniques such as but not limited to mass spectrometry, HPLC, etc.

Polypeptide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

An informal sequence listing is appended to this application and appears after the Examples section.

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning-A Laboratory Manual, Cold Spring Harbor Laboratory, Cold Spring Harbor, New York; Wetmur, 1991, Critical Review in Biochem. and Mol. Biol. 26(¾):227-259; and Owczarzy et al., 2008, Biochemistry, 47: 5336-5353, which are incorporated herein by reference).

The terms “nucleic acid” and “nucleic acid molecule,” as used herein, refer to a compound comprising a nucleobase and an acidic moiety, e.g., a nucleoside, a nucleotide, or a polymer of nucleotides. Nucleic acids generally refer to polymers comprising nucleotides or nucleotide analogs joined together through backbone linkages such as but not limited to phosphodiester bonds. Nucleic acids include deoxyribonucleic acids (DNA) and ribonucleic acids (RNA) such as messenger RNA (mRNA), transfer RNA (tRNA), etc. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

Nucleic acids, proteins, and/or other compositions described herein may be purified. As used herein, “purified” means separate from the majority of other compounds or entities, and encompasses partially purified or substantially purified. Purity may be denoted by a weight by weight measure and may be determined using a variety of analytical techniques such as but not limited to mass spectrometry, HPLC, spectrophotometer, etc.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “5′-C-A-G-T,” is complementary to the sequence “5′-A-C-T-G.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

In the context of the present disclosure, the following abbreviations for the commonly occurring nucleic acid bases are used. “A” refers to adenine, “C” refers to cytosine, “G” refers to guanine, “T” refers to thymine, and “U” refers to uracil. The aforementioned abbreviations may also be used to refer to nucleosides or nucleotides comprising the nucleic acid bases. For example, “G” may refer guanine, guanosine, or guanidine, depending on the context.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of′ should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

### EXAMPLES

The following Examples are illustrative and should not be interpreted to limit the scope of the claimed subject matter.

### Example 1: High-Avidity Binding Drives Nucleation of Amyloidogenic Transthyretin Monomer

Construction of biotin-TTR fusion and SA-inducedpolymer. The inventors constructed full-length human TTRWT, V30M, and V122I proteins each with an N-terminal AviTag, which was subjected to site-directed biotinylation catalyzed by BirA enzyme (FIG. 1, A and B). Via tight binding between the biotin tag and tetrameric streptavidin (SA), larger TTR complexes were formed in conjunction with SA as determined by SDS PAGE (FIG. 1C). A TTR-SA lattice structure (modeled in FIG. 1B) included 20-40 TTR subunits per induced complex as measured by size-exclusion chromatography (SEC) (FIG. 1D and FIG. 8).

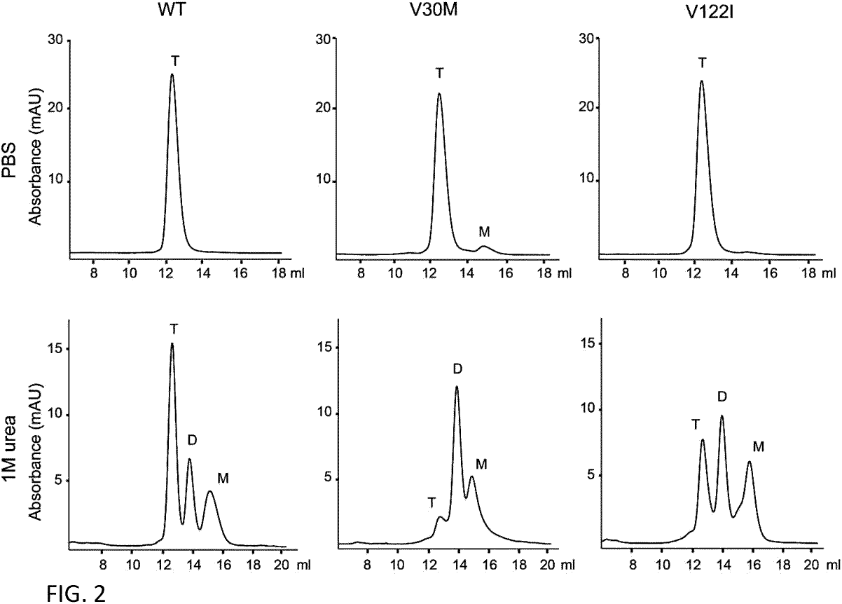

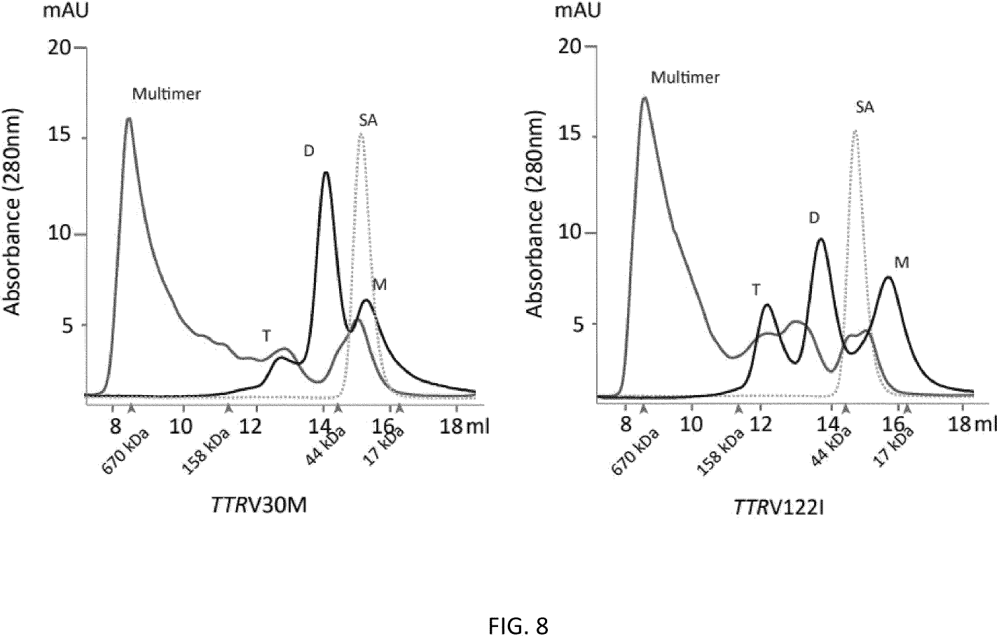

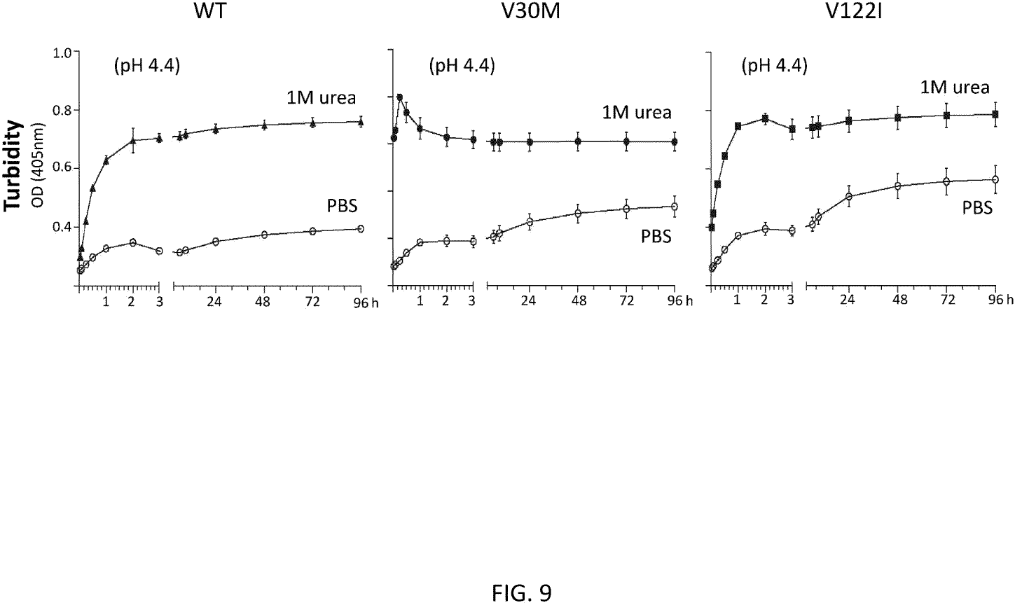

Distinct distributions of TTR monomer, dimer, and tetramer contents among WT, V30M, and V122I at pH 7.6 in 1 M urea. Without induction by SA, WT, V30M, and V122I appeared as predominantly stable tetramers at pH 7.4 in PBS (FIG. 2). As low pH conditions were known to promote disassembly of TTR tetramers into monomers (39, 40), the inventors also characterized the variants at pH 4.4 in either PBS or 1 M urea solution. Switching to pH 4.4 caused instant formation of protein precipitation. Over a longer period, there was further increase of turbidity (FIG. 9), indicating formation of large protein aggregates in keeping with previous reports (40, 41).

Visible protein precipitation usually contains thousands of protein subunits in amorphous aggregates (42, 43). The inventors were more interested in earlier formation of soluble oligomers comprised of only dozens of protein subunits. To this end, the inventors followed an alternative workflow that first involved the denaturing of protein in 8 M urea, followed by gradual renaturing through dialysis using 1 M urea at pH 7.6. Upon analysis by SEC, TTRWT, TTRV30M, and TTRV122I showed very different distributions among their monomer, dimer, and tetramer contents (FIG. 2). TTRWT had the highest tetramer level among the variants, with the lowest level of monomers. In contrast, TTRV30M dimer formed a tall peak, in keeping with the understanding of V30M’s propensity to form aberrant intermolecular disulfide bridges (13, 44, 45). TTRV122I had a relatively “balanced” tetramer, dimer, and monomer distribution, with the highest level of monomeric form compared with its WT and V30M counterparts. These results, despite being obtained from an unnatural process of protein renaturing, were generally in agreement with the expectation of TTRWT being most stable, in contrast to TTRV30M and TTRV122I that showed the propensity of adapting dimeric and dimeric/monomeric amyloidogenic folds, respectively.

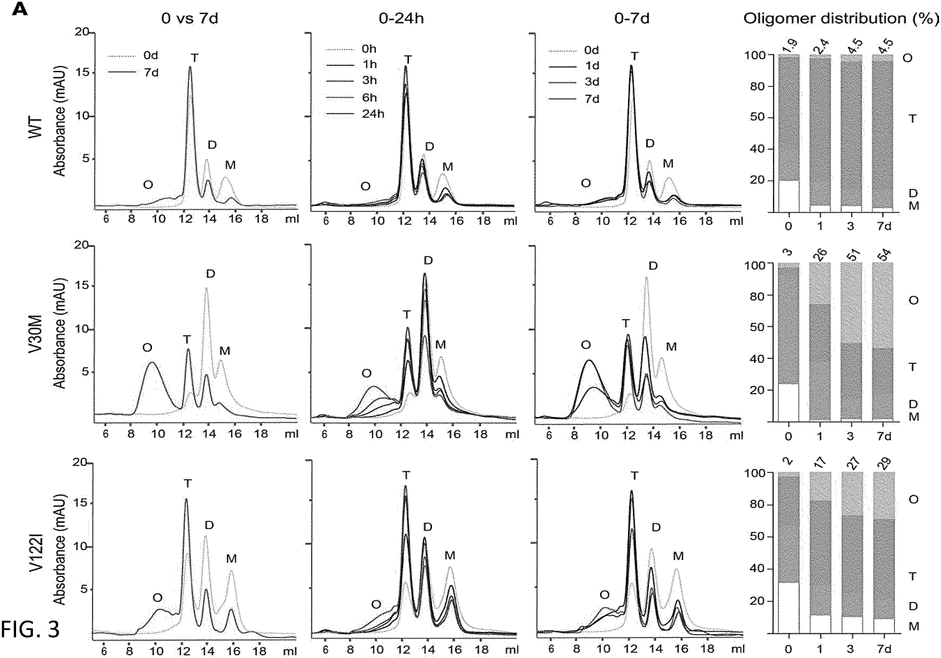

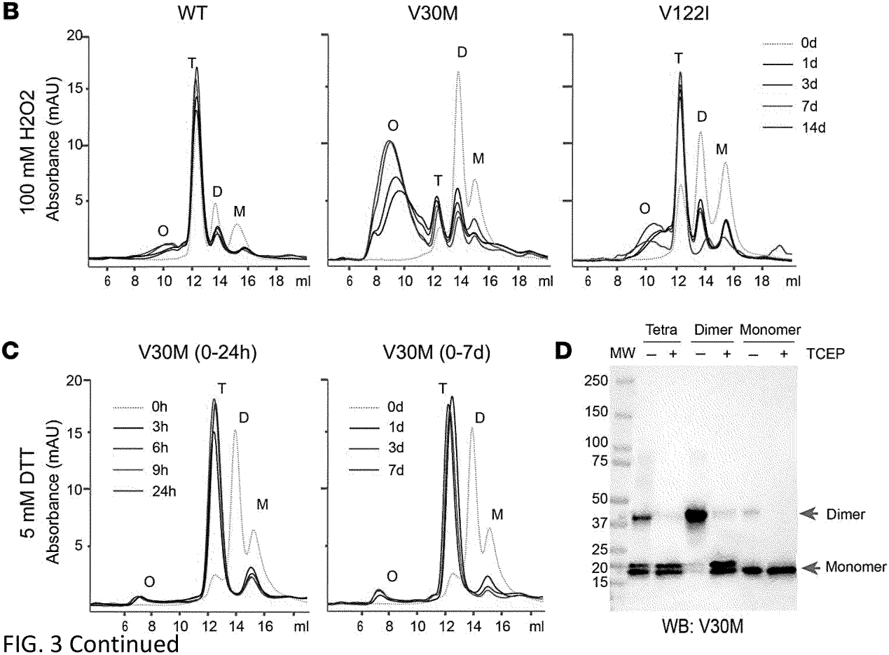

Distinct dynamics in time-dependent oligomeric transformation among TTR variants of WT, V30M, and V122I. Next, the inventors set out to measure early oligomeric transformation of those TTR variants in 1 M urea at pH 7.6 by running SEC. Freshly thawed proteins were placed at room temperature on a rotating platform for up to 7 days. Aliquots were collected from the reaction in a time series from 1 hour to 7 days and subsequently analyzed by SEC (FIG. 3A). In addition to the 3 distinct peaks in the lower molecular range of monomers, dimers, and tetramers, a UV-absorption reading of protein gradually increased in the higher molecular range between 200 and 600 kDa (FIG. 10). These intensities reflected the time-dependent accumulation of TTR oligomers in 1 M urea solution.

Among the variants, WT TTR had the mildest increase in the amount of oligomers (FIG. 3A). It was also noted that the peak heights for WT monomers and dimers gradually decreased, whereas the tetrameric content increased only slightly. It is conceivable that while the tetrameric form of WT TTR was stable, the accumulation of new TTR oligomers was largely assembled from WT monomers and possibly dimers as well. In contrast, TTRV30M showed the largest accumulation of its oligomer signals during the same period of time (FIG. 3A). Meanwhile, the heights of both dimer and monomer peaks were greatly reduced. Unexpectedly, the tetrameric amount substantially increased, which could only be explained by the contribution of refolding from TTR monomers or dimers, or both concurrently (FIG. 3A). TTRV122I, which had prominent dimer and monomer peaks at the beginning, showed a modest increase of oligomer levels (comparisons of AUC in FIG. 3A) that were greater than that of WT but substantially smaller than that of V30M.

It is interesting to note that in addition to the comparisons of AUC for total oligomer contents, there were clear distinctions in the average molecular size of TTR polymers among the variants (FIG. 10). TTRWT had the smallest average polymer size of approximately 200 kDa, the equivalent of approximately 10 protein subunits; TTRV122I had its average polymer size of approximately 300 kDa, the equivalent of approximately 15 protein subunits; whereas TTRV30M had its average polymer size of approximately 500 kDa, the equivalent of approximately 25 protein subunits. However, this trend of polymer size was only associated with variant types, regardless of the length of incubation time (FIG. 3A), indicating that each variant adapted a distinct assembly unique to the type of mutation and the associated structural fold.

Oxidative condition further accelerated TTRV30M’s oligomeric transformation. Next, the inventors examined the effect of 100 µM H₂O₂ on these variants in 1 M urea at pH 7.6 (FIG. 3B). Both TTRWT and TTRV122I had relatively moderate increases of their oligomeric contents over a period of 14 days compared with the condition without H₂O₂ treatment (compare FIG. 3, A and B). In contrast, TTRV30M had a greater accumulation of its high molecular weight oligomers after being treated with H₂O₂ (FIG. 3B). There was also a further increase of the average molecular size of V30M oligomer contents over time. These results were largely in agreement with the understanding of TTRV30M being able to form an aberrant intermolecular disulfide bridge. Conversely, treatment of TTRV30M with reducing agent Dithiothreitol (DTT) at 5 mM in 1 M urea and pH 7.6 completely abolished dimeric forms, partially reduced the level of monomers, but greatly restored tetramer content with an increase of more than 10 folds in its level over time (FIG. 3C). To further investigate the disulfide bridge formation in TTRV30M, the inventors separately isolated SEC fractions of monomers, dimers, and tetramers, and then subjected the fractions to SDS PAGE. Under the nonreducing condition, the V30M dimer appeared to be coupled exclusively by disulfide bridge because Tris (2-carboxyethyl) phosphine (TCEP) converted all dimers to monomers (FIG. 3D). In contrast, the tetramers contained only a small number of disulfide-linked dimers in the presence of predominately noncovalently linked subunits (FIG. 3D) that were expected to adopt their normal tetrameric fold. These results indicated that the instability of TTRV30M was largely driven by aberrant intramolecular disulfide connections that favored a dimeric configuration.

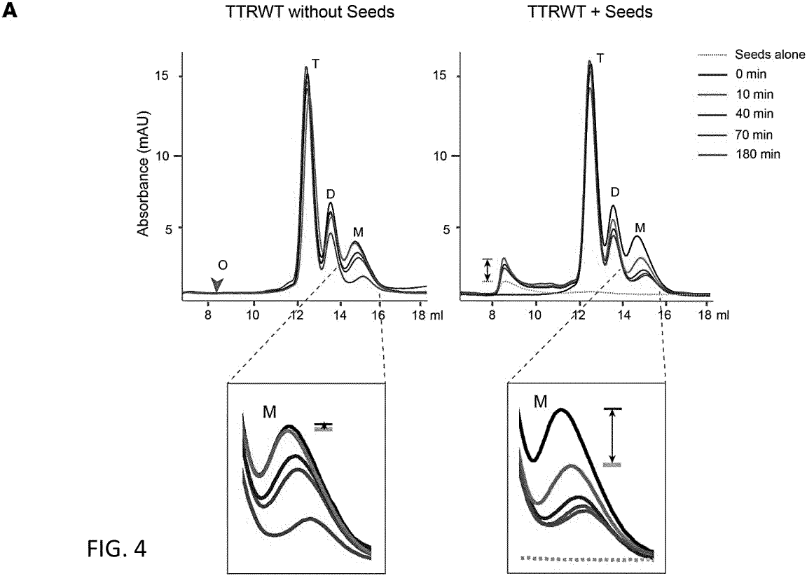

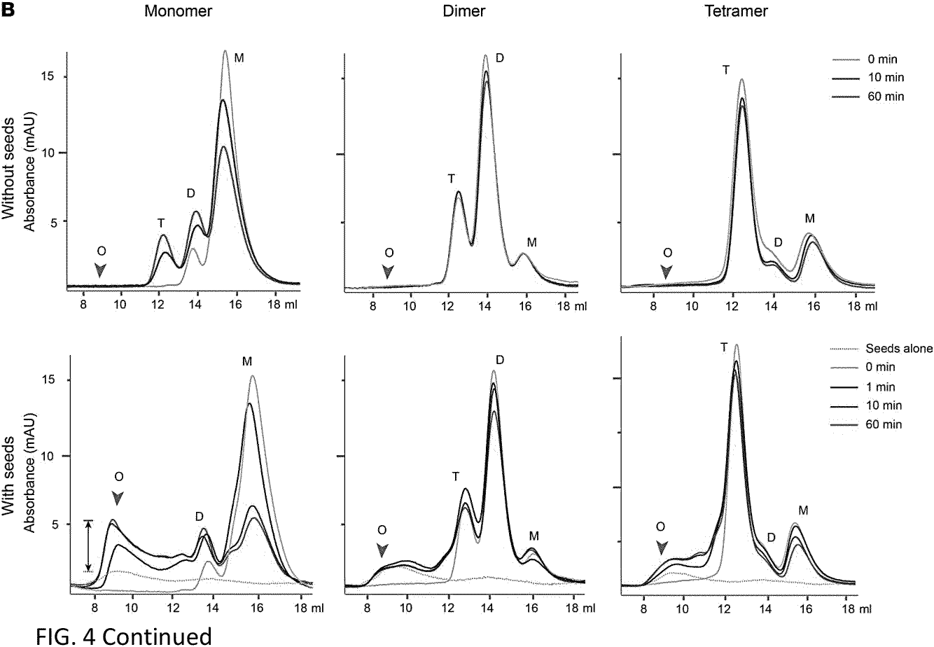

Artificially induced multimeric seeds capable of depleting TTR monomers and dimers within minutes. The inventors asked about potential seeding effects on folding energetics that promote TTR oligomeric aggregation. The inventors were particularly interested in whether amyloid seeding can accelerate oligomerization of monomers and dimers. To this end, the inventors added a substoichiometric amount of SA-induced TTRV30M multimer as the seed to TTRWT in 1 M urea buffer. The inventors then measured the changes of TTR complex size in a time series (FIG. 4). It should be noted that this SA-induced TTR multimer was not expected to structurally resemble TTR oligomers that adapt β-sheet stacking (15). In theory, the artificial aggregation of TTR through SA-mediated interaction with the N-terminal biotin tag results in a greater number of exposed DAGH sheets available for nucleating soluble TTR monomers (schematics in FIG. 1B).

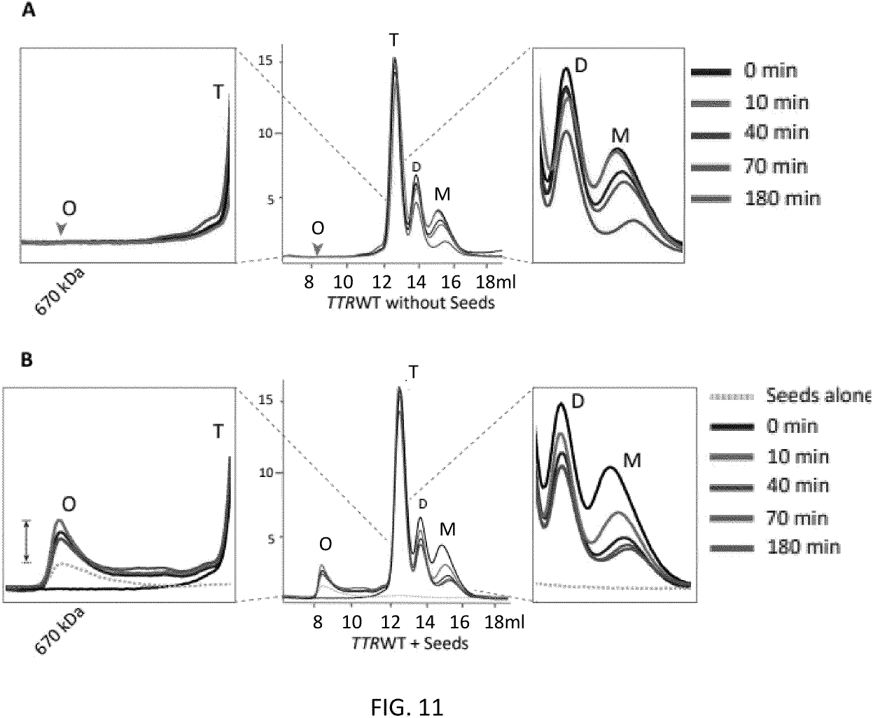

Following the “spike-in” of SA-V30M multimers to freshly thawed TTRWT, there was a rapid reduction of the monomer peak (FIG. 4A). To a lesser degree, TTRWT dimers were also reduced in response to the seeds, whereas the tetramer peak height remained relatively unchanged. Meanwhile, within 10 minutes, the high molecular weight of the approximately 600 kDa content rapidly increased (FIG. 4A and FIG. 11), which was likely attributable to an induced aggregation of monomers and dimers toward the SA-V30M seed. It is important to note that the overall molecular size of seed-induced complexes was larger than that of spontaneously self-aggregated TTRV30M (FIG. 4A compared with FIG. 3A). TTRWT in the absence of the seeds showed little increase in oligomers. Instead, the slow reduction of its monomer and dimeric contents resulted in an increase of tetramers, suggesting a process of monomers adapting correct refolding into tetramers (FIG. 4A and FIG. 11). In contrast, in the presence of the artificial inducer of SA-V30M as seeds, TTR monomers rapidly coalesced with the seeds instead of refolding into tetramers (FIG. 4A and FIG. 11).

Next, the inventors focused on the response of TTRWT monomers to the seeds in the absence of possible interference by other TTR forms. First, by running SEC, the inventors isolated the monomers and subjected them to conditions either with or without SA-V30M seeds (10% w/w) (FIG. 4B). In the absence of the seeds, TTR monomers gradually reduced their levels while there was an increase of both dimeric and tetrameric contents without formation of oligomeric TTR (FIG. 4B). In contrast, following the spike-in of the seeds, within the first 10 minutes) there was already a drop of monomer levels with a rapid increase of high molecular weight oligomers (FIG. 4B). Meanwhile, there was little change in dimer and tetramer levels, suggesting a potent nucleation ability of the artificial seeds towards TTR monomers. In parallel, the inventors also isolated dimeric and tetrameric TTRWT and subjected them to reactions with or without SA-V30M seeds. Unlike the rapid response of monomeric TTRWT to seeding, the tetramers and dimers were not affected after multimer seeds were added (FIG. 4B). It is also interesting to note that despite the rapid drop of monomer contents after reaction with seeds between 0-10 minutes, the remaining monomer levels between 10-60 minutes changed very little. This suggested the presence of a mixture of misfolded TTR monomers that responded to the seeds and a smaller fraction of stably folded TTR monomers that was resistant to aggregation, which is consistent with the persistence of monomer contents associated with dimer and tetramer fractions.

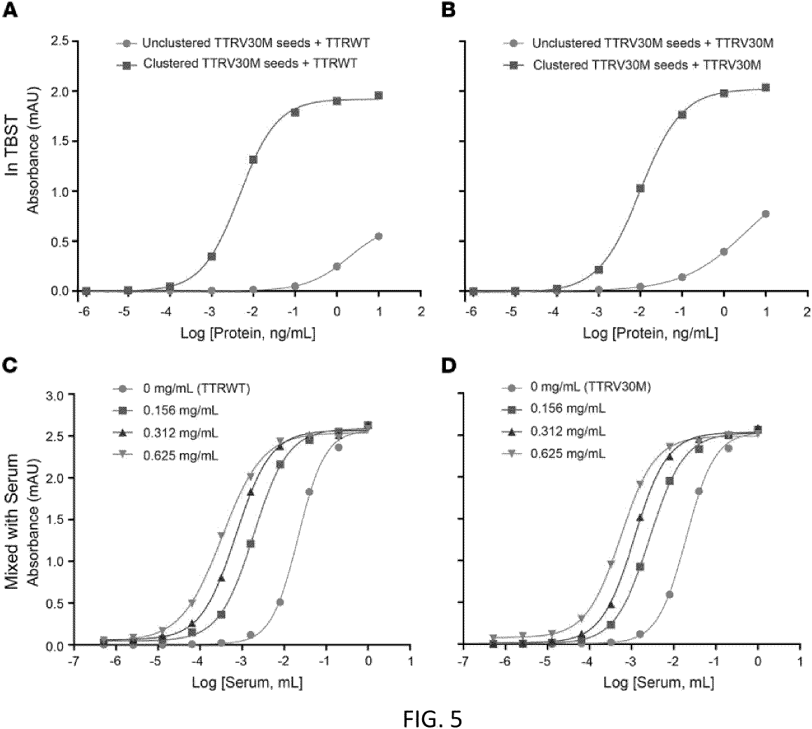

New ELISA kit based on the high valency of poly-TTRV30M multimer for detecting misfolded TTR. Next, the inventors wanted to exploit the high valency of synthetic poly-TTR seeds in selective binding of structurally unstable, and thus potentially amyloidogenic, TTR. Instead of measuring coalescence of TTR by the poly-TTRV30M seeds in solution (FIG. 4), the inventors immobilized the seeds on ELISA plates as capturing probes (FIG. 5), as described in Methods. Separately, the inventors prepared the “prey” proteins of recombinant WT and V30M TTR with Tandem Mass Tags (TMT) to facilitate their detections by an anti-TMT antibody (Thermo Fisher). In contrast to uninduced TTRV30M, SA-induced poly-TTRV30M-coated wells captured TMT-labeled WT and V30M TTR in TBS Tween (TBST) (FIG. 5, A and B). Next, the inventors explored the ELISA kit for detecting serum proteins that can be captured by these poly-TTRV30M seeds (FIG. 5, C and D). First, the inventors labeled pooled human sera with TMT and divided the sample in a dilution series to be incubated with immobilized seeds. As expected, serum protein signals were detected with the seed-coated ELISA plate. To further ascertain that misfolded TTR in serum can be detected by the method, the inventors spiked-in recombinant TTRWT and TTRV30M proteins that had been partially destabilized in 1 M urea as positive controls. As expected, the ELISA kit could detect the spiked-in TTR controls (FIG. 5, C and D). These results provided the initial validation of poly-TTRV30M seeds-based methodology for measuring potentially amyloidogenic TTR contents in serum, although the inventors caution that further characterization of the ELISA kit is needed by using confirmed ATTR samples.

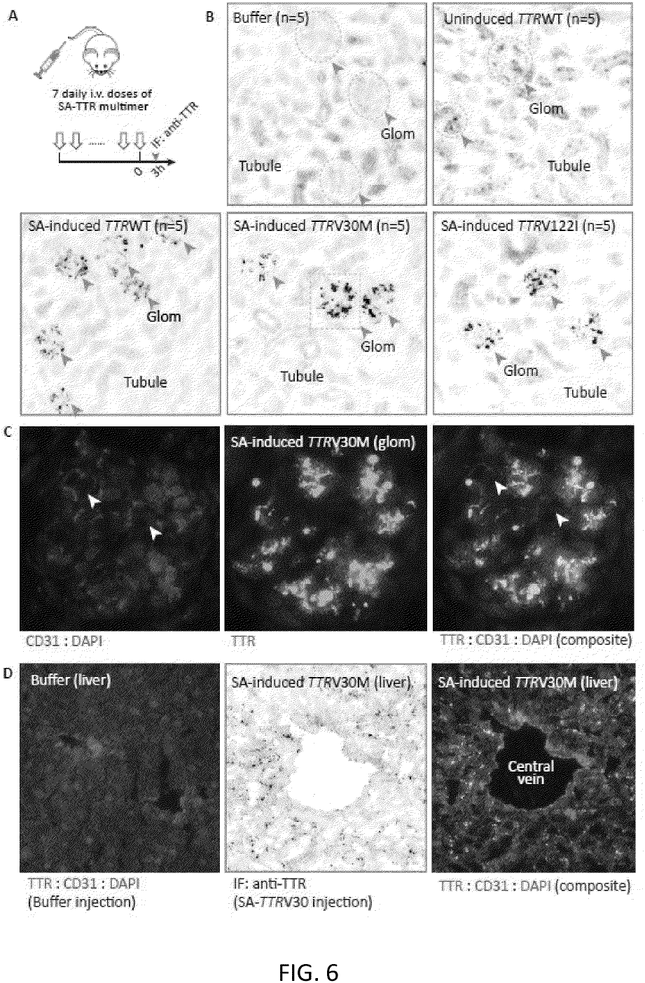

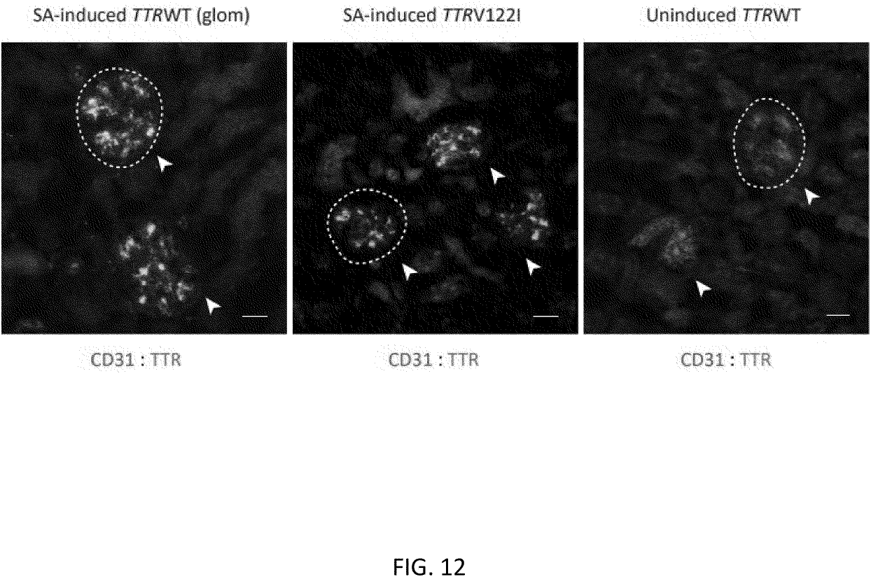

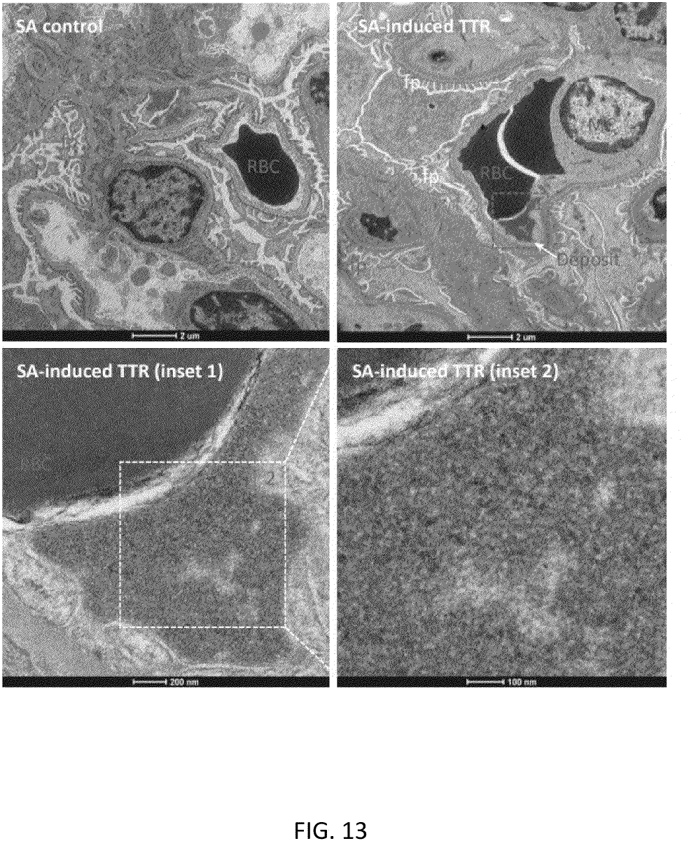

Intravenously injected SA-TTR multimer-formed renal deposits in mice. Considering the nucleation capability of this SA-induction model, the inventors sought to determine possible in vivo effects of the induced complexes. To this end, the inventors directly injected SA-induced multimers in mice at 15 mg/kg BW (FIG. 6A). Each group was assigned 5 mice to receive 7 daily injections of TTRWT, TTRV30M, or TTRV122I. Organs such as the heart, the liver, and the kidney were harvested 3 hours after the last injection and tissue specimens were probed for TTR using immunofluorescence (IF). Few tissue deposits were formed by uninduced recombinant TTRs (FIG. 6B and FIG. 12). In contrast, prominent TTR deposition was observed in the kidney glomerulus of all mice that received the injection of SA-induced TTRs (FIG. 6B and Table 1). Co-staining of the specimens of vascular endothelium marker CD31 showed TTR deposits outside of the capillary lumen (FIG. 6C). It is, therefore, conceivable that the deposits were formed after TTR had exited blood circulation and entered glomerular mesangial areas in the kidney. Furthermore, TTR puncta in the kidney appeared to vary greatly in size and shape (FIG. 6, B and C), possibly attributable to further growth of TTR aggregates in renal interstitium. Transmission electron microscopy (TEM) images of the glomerulus revealed electron-dense amorphous deposits in the mesangium (FIG. 13), indicating the formation of nonfibrillar oligomeric intermediates. In the liver, SA-induced TTRV30M also formed deposits (FIG. 6D), which were consistent with the general function of the liver in clearing large protein complexes from circulation.

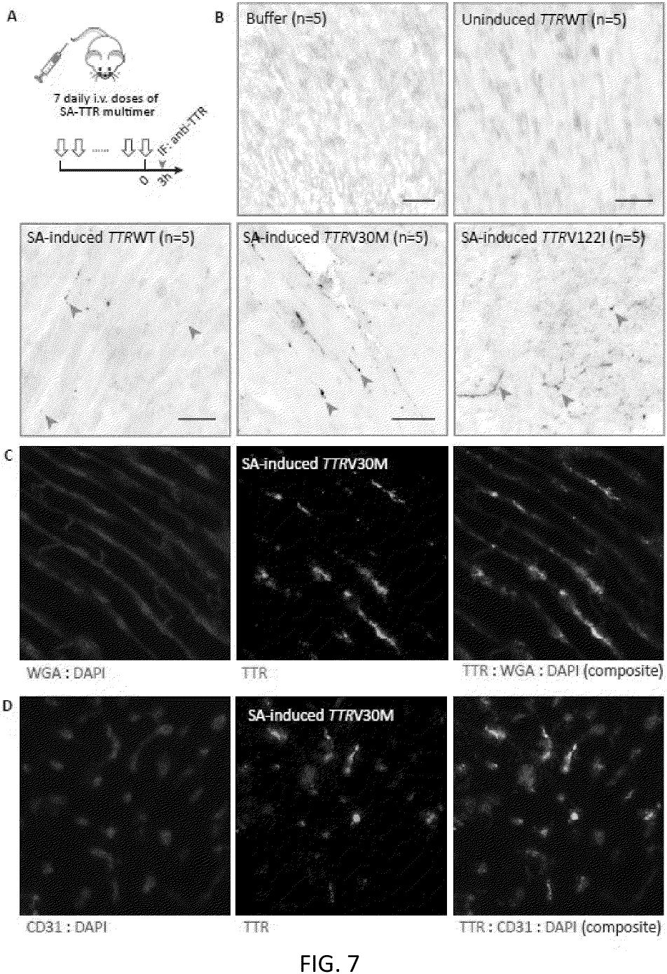

Intravenously injected SA-TTRV30M multimer-formed prominent cardiac deposits in mice. In contrast to the kidney that developed deposits from SA induction regardless of the type of TTR variants, some, but not all, mice in each injection group had cardiac deposits after 7 daily doses of the injection (FIG. 7A and Table 1). There were also far fewer TTR deposits in the heart in terms of fluorescence intensity and prevalence. Among the 3 variants induced with SA, TTRV30M had by far the strongest fluorescence intensity and a broader presence of deposition in the heart, whereas WT and V122I deposits were sparse (FIG. 7B).

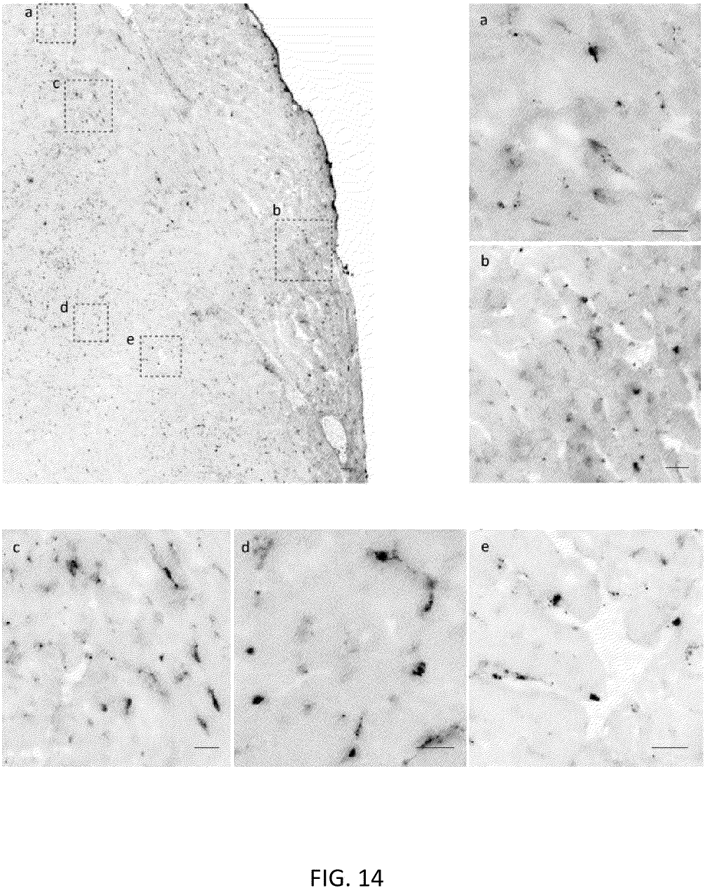

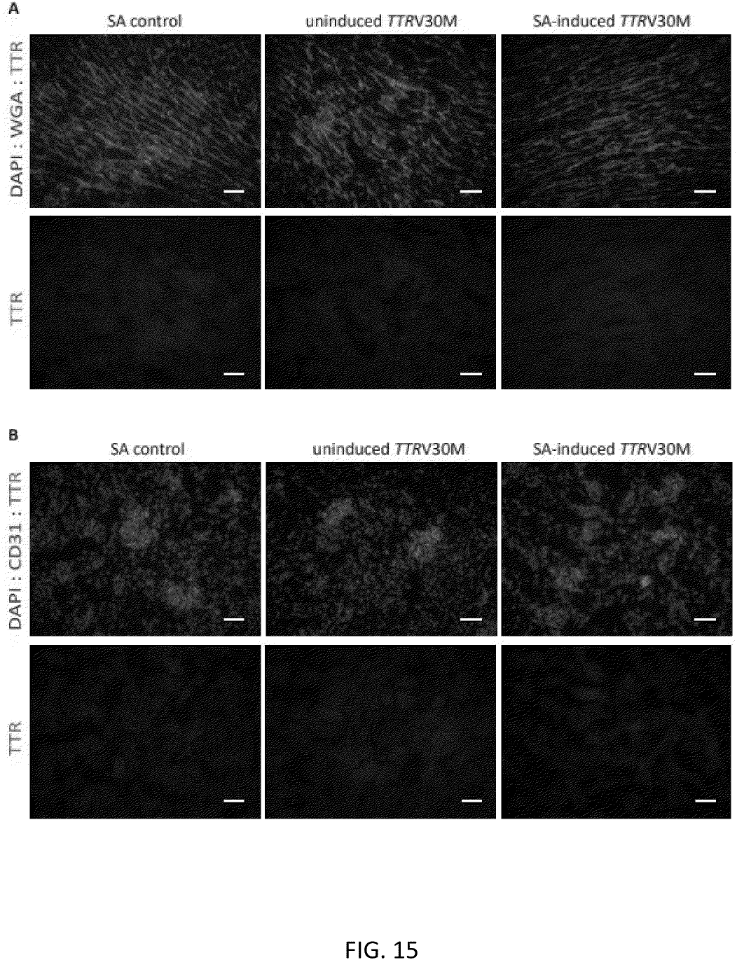

From TTRV30M injection, most deposits were in the myocardium layer (FIG. 14), reminiscent of chronic deposition in ATTR-CM. Longitudinal sections showed the distribution of TTRV30M deposits located primarily in the intermyofibrillar space along the sarcolemma (FIG. 7C). Cross sections showed the deposits associated with myocardial vessels (FIG. 7D), likely in the interstitial space between the sarcolemma and the capillary. Overall, these TTR deposits varied in size and fluorescence intensity in highly clustered patterns (FIG. 7 and FIG. 14). It should be noted that 4 weeks after the last injection, TTR deposits were no longer detectable in the heart or in the kidney (FIG. 15).

In this work, the inventors focused on comparing the dynamics of TTR transitions among monomeric, dimeric, tetrameric, and early intermediates and polymeric states. The study was performed in the context of common TTR variants of WT, V30M, and V122I in the presence or absence of a synthetic TTRV30M multimer as the seed for nucleating TTR. The key innovation of the study was the construction of TTR with an N-terminus biotin tag that, through interacting with SA, robustly assembled TTR into 20-40 subunit lattices. This induced TTRV30M multimer lattice functioned as a high avidity trap to recruit TTR monomers and dimers within minutes, while more stable TTR tetramers were unaffected by the multimeric trap. Our observations were consistent with what Saelices et al. previously showed using ATTR fibrils extracted from patients’ heart tissues to accelerate monomeric TTR aggregation ex vivo (24). In keeping with the observed activity of our synthetic TTR multimer to specifically coalesce misfolded monomers, immobilized synthetic multimers devised as the capture reagent in an ELISA kit were used to measure serum contents of misfolded TTR species. In addition, our artificially induced TTRV30M multimer also demonstrated an in vivo capability of forming ATTR-like cardiac deposits in mice that possibly resemble TTR seeding in early stages of ATTR clinical development. To our limited knowledge, our system was the first cardiac model of acute ATTR deposition. Compared with the transgenic mouse method (46, 47) that takes almost 2 years to develop cardiac deposits, our injection model can quickly generate the phenotype with an extensive display of ATTR-like deposits in the myocardium that likely resemble early events of amyloidosis.

Extraction of TTR fibrils from ATTR hearts inevitably contains a large amount of non-TTR proteins and requires healthy tissue controls (24, 48). In contrast, our synthetic TTR multimers from recombinant production were more homogenous. As measured by size-exclusion chromatography, more than 90% of TTRs were assembled into higher-order multimers following SA induction (FIG. 1D and FIG. 8). The inventors should emphasize that these SA-TTR multimers were assembled from a mixture of TTR monomers, dimers, and tetramers. In conjunction with the tetrameric state of SA, SA-TTR complexes were expected to adapt a lattice-like configuration (FIG. 1B), consisting of an estimated 20-40 TTR subunits as determined by the combined sizes of the complexes (FIG. 1D). Structural analysis by Saelices et al. specifically identified β-strands that are more exposed in TTR monomers than in tetramers to explain how monomers are prone to primary nucleation (49). In our SA-TTR lattices, TTR proteins were in spatial vicinity to each other without adapting a cross-β spine configuration of mature amyloid fibril. It is conceivable that the high density of adhesive surfaces presented by the cluster of individual monomers and dimers within the SA-TTR complex could rapidly nucleate soluble monomers due to an avidity effect. Although amyloid deposits in tissues are predominantly bundled fibrils, each in a zipper spine configuration, the combined fibril ends may still present an avidity advantage to recruit soluble TTR monomers. In amorphous amyloid aggregates, which the artificial SA-TTR complexes may resemble, the ability to recruit soluble TTR might also be attributable to presumed high avidities. A prior study by Saelices et al. (49) showed strong seeding effects at physiological pH 7.6 of either cardiac fibrils or in vitro constructed monomeric TTR (“MTTR”) aggregates that Jiang et al. first introduced (50).

It is of particular note that TTR oligomerization is a complex and still partially understood process and may even follow distinct assembly pathways. Pathogenic mutations are structural factors for aberrant fibrillar assembly that can cross-seed WT TTR. Environmental conditions can also influence the conformational flexibility of TTR monomers in forming morphologically different oligomers (26, 36, 51). TTR oligomerization is driven by monomer misfolding and monomer-monomer interactions via solvent-exposed proamyloidogenic surfaces (50). Nevertheless, oligomerization is a dynamic process of complex monomer-monomer, monomer-oligomer, and oligomer-oligomer interactions, which also involve adaptive conformational changes of the terminal TTR subunits in growing prefibrillar aggregates. Furthermore, tissue tropism and cytotoxicity also contribute to the ultimate formation of mature amyloid fibrils in disease.

Again, it should be clarified that the 1 M urea condition at pH 7.6 by no means resembles the natural environment. The inventors favored the condition only because it greatly eases the transformation among monomeric, dimeric, tetrameric, and oligomeric forms, whereas PBS at pH 7.6 stabilizes tetramers (FIG. 2) and a low pH condition causes quick disassembling of tetramers and amyloid fibril formation (39, 40). Indeed, among TTRWT, TTRV30M, and TTRV122I, the 1 M urea condition showed clear distinctions among the variants in terms of the distribution of monomer, dimer, and tetramer contents (FIG. 2). Therefore, the 1 M urea condition that seemed to amplify the destabilizing effects was ideal for our analytical purposes.

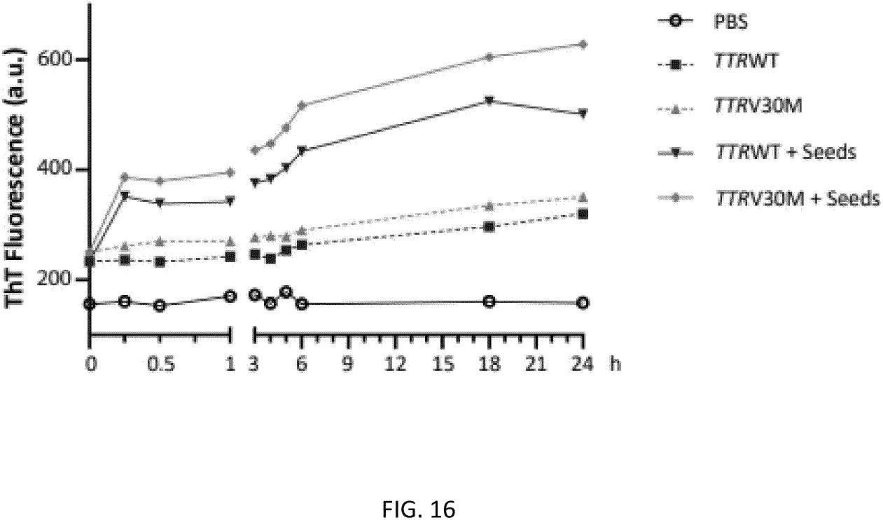

Unlike the endogenous process, our workflow of renaturing TTR variants into their folded states greatly boosted the aberrant monomer and dimer contents. In human cells, the endoplasmic reticulum-Golgi not only has chaperones to assist protein folding, but also has intrinsic quality control steps to further prevent the secretion of misfolded TTR (52-54). The actual levels of misfolded TTR relative to the levels of properly folded tetramers in blood are expected to be very low, even in patients with ATTR. The slow progressive nature of the disease in patients and in mouse models has greatly limited research advancement. Our recombinant multimers represent an attractive alternative in which phenotypes of TTR deposition in the heart can be achieved in days by injecting artificially aggregated SA-TTR complexes. However, it should be clarified that the organ deposits of our induced TTR multimers did not resemble natural amyloid fibrils of cross-β-sheet assembly. Our ex vivo results showed a strong propensity of the artificial complex to nucleate TTR monomer, a characteristic shared with ATTR oligomeric precursors. However, although our synthetic high-order TTR seeds could rapidly nucleate misfolded monomers, there was only a modest increase of thioflavin T (ThT) signals (FIG. 16), indicating the assembly of TTR subunits within the oligomeric TTR lattice was not in the form of long amyloid fibrils. This is consistent with the lack of green birefringence signals from Congo red staining of the heart and the kidney under polarized light (not shown). Future studies need to address the long-term consequence of whether the artificial SA-TTR complex can cross-seed the nucleation of endogenous TTR in vivo.

Construction of recombinant TTR with AviTag. Full-length human TTR (Uniprot, P02766) was used as the template for constructing recombinant TTRWT protein. Encoding cDNA was synthesized by Integrated DNA Technologies with codons optimized for E. coli expression. This TTR cDNA was fused to an N-terminus AviTag sequence (encoding amino acids GLNDIFEAQKIEWHE, SEQ ID NO: 5). The fusion was cloned into a PET30a vector (Invitrogen) with the addition of a 6xHis purification tag. TTRV30M and TTRV122I variants were made by mutagenesis to the WT template.

Recombinant protein production and purification. Recombinant TTR expression was induced with 0.3 mM isopropyl-β-d-thiogalactoside for 16 hours at 25° C. Bacterial pellets were collected by centrifugation and then stored at -80° C. On the day of purification, the bacterial pellet was resuspended in 0.13 M NaCl, 20 mM Na₂HPO₄ buffer (pH = 7.4) supplemented with 0.5 mg/mL lysozyme for 30 minutes followed by sonication; the mixture was then subjected to centrifugation. The clear supernatant that contained AviTag-TTR was loaded onto a Histrap column (GE Healthcare), and the recombinant protein was collected with elusion buffer containing 250 mM imidazole. Imidazole was then removed by desalting and buffer exchange, adjusted to PBS pH 7.4. The final protein concentration was calculated using a BCA kit (Pierce) and the protein purity was determined by SDS-PAGE analysis. TTR in 1 M urea from protein renaturing treatment was prepared from the inclusion body. After lysis, the bacterial pellets were collected and then washed in 1 M urea with PBS (pH 7.6). The resulting pellets were lysed and resuspended in 8 M urea buffer (pH 7.8) at 4° C. for 24 hours. Following an additional centrifugation, supernatant was loaded onto a Histrap column. Denatured TTR was collected following elution using imidazole. Renaturing was performed by dialysis with 1 M urea pH 7.6 overnight. Biotinylation and SA-induced multimerization were performed in the 1 M urea buffer.

Site-directed biotinylation of TTR to an N-terminus AviTag and multimeric induction of biotin-TTR with SA. BirA biotin ligase was produced using a BL21 (DE3) expression system. Purified AviTag-TTR proteins were subjected to site-directed biotinylation in 0.2 mM ATP, 5 µM MgCl2 together with BirA (55). Each TTR polypeptide was labeled with 1 biotin group attached to the AviTag sequence. To induce multimerization of biotin-conjugated TTR, SA (Agilent Technologies) was added at approximately a 1:4 molar ratio. Unincorporated free biotin was removed by desalting column (GE Healthcare).

Size-exclusion chromatography and gel analyses of TTR multimericity. Preparational and analytical size-exclusion chromatography was performed on Superdex 200 Increase 10/300 GL (GE Healthcare). Time series analyses were conducted by running sample aliquots on the same column in succession approximately 30 minutes apart. Gel analyses for estimating the molecular size of TTR complexes were performed by comparing reducing versus nonreducing conditions with or without 50 mM TCEP, respectively. It should be noted that biotin-SA interactions can withstand 1% SDS in sample buffer for SDS PAGE. Reducing condition in the presence of 50 mM TCEP disrupts biotin-SA interactions as well as disulfide bonds between TTR subunits. ThT assay was performed in FLUOTRAC 96-well microtiter plates in 25 µM ThT in phosphate saline and 1 mg/mL of uninduced TTR with or without 0.1 mg/mL of streptavidin-induced (SA-induced) TTRV30M seeds at 37° C. Fluorescence signals at 450 nm excitation/485 nm emission were read at indicated time points. For measuring solution turbidity, WT, V30M, and V122I samples at 1 mg/mL concentration were stored in PBS or 1 M urea buffer. In a 1:1 ratio (v/v), the samples were then mixed with pH 4.4 solution of 100 mM acetate sodium, 100 mM KCl, and 1 mM EDTA. Turbidity was measured at 405 nm over 96 hours at 25° C. All experiments were repeated 3 times.

TTR seeding using SA-induced TTRV30M on TTRWT. Multimers of SA-induced TTRV30M seeds were prepared by collecting SEC elution fractions between 8-10 mL of the SA-TTRV30M reaction. The seed concentration was then adjusted to 0.1-0.12 mg/mL. The seeds were mixed with recombinant TTRWT (1 mg/mL in 1 M urea buffer) or its monomer/dimer/tetramer isolates in a 1:10 (w/w) ratio (56). Aliquots were analyzed on the same column approximately 30 minutes apart in succession. All experiments were repeated 3 times.

Construction of poly-TTRV30M-based ELISA and assay development for detecting serum contents of unstable TTR. Coating of the microtiter plates was performed with 100 µL of either TTRV30M recombinant protein or SA-induced TTRV30M multimers at 5 µg/mL concentration in each well at 4° C. overnight. Following washing with TBST, the plates were blocked using 1% BSA. For labeling either purified TTR proteins or whole serum with TMT (Thermo Fisher), 800 µg of recombinant protein or 100 µL of serum was desalted (using Pierce desalting spin column) and then incubated with 400 µg TMTzero reagent for 3 hours at room temperature following manufacturer’s standard protocol. The reaction was quenched by the addition of Tris buffer, followed by desalting. Then, in a dilution series, 100 µL of TMT-labeled samples was incubated in the precoated wells for 1 hour at room temperature. Following additional washing steps with TBST, anti-TMT monoclonal detection antibody (Thermo Fisher, 25D5) at 1:1000 dilution and HRP-conjugated anti-mouse IgG secondary antibody were used to detect levels of proteins coaggregated with the multimer TTR bait.

Injection of recombinant TTR complexes in mice. BALB/cJ strain of mice (Charles River Labs) between 16-20 weeks of age were separated into groups with an approximate matching distribution of sex and weight. For 7 consecutive days 300 µg of TTR protein was injected via the tail vein. The animals were then sacrificed and the organs, including the heart, the kidney and the liver, were collected 3 hours after receiving the last dose of injection. The specimens were minced into smaller pieces to be embedded in OCT (Thermo Fisher). Frozen tissues were sectioned at 4 µm thickness for staining using anti-TTR antibody (Agilent Technologies, A0002) at 1:200 dilution, and goat anti-rabbit IgG Alexa-488 (Novus Biologicals, A11034) at 1:400. Counterstains used anti-mouse CD31 antibody (BD Pharmingen), wheat germ agglutinin (WGA) Alexa Fluor conjugate (Thermo Fisher) at 1:100 dilution, and DAPI (Sigma-Aldrich). The method for transmission electron microscopy of kidney deposits was described previously (57).

TABLE S1

Summary of mouse phenotypes following injection of streptavidin-induced TTR multimer

| Experimental group | Number of mice | TTR deposition in kidney | Average renal TTR score by IF | TTR deposition in heart | Average heart TTR score by IF |
|---|---|---|---|---|---|
| WT+SA | 5 | 5/5 | +++ | ⅖ | + |
| V122I+SA | 5 | 5/5 | +++ | ⅖ | ++ |
| V30M+SA | 5 | 5/5 | +++ | ⅖ | +++ |
| WT only | 5 | ⅕ | + | 0/5 | - |
| PBS+1M Urea | 5 | 0/5 | - | 0/5 | - |

### References

1. Schmidt HH, et al. (2018) Estimating the global prevalence of transthyretin familial amyloid polyneuropathy. Muscle Nerve 57(5):829-837.

2. Damrauer SM, et al. (2019) Association of the V 122I Hereditary Transthyretin Amyloidosis Genetic Variant With Heart Failure Among Individuals of African or Hispanic/Latino Ancestry. JAMA 322(22):2191-2202.

3. Sinha A, et al. (2020) Association of the V122I Transthyretin Amyloidosis Genetic Variant With Cardiac Structure and Function in Middle-aged Black Adults: Coronary Artery Risk Development in Young Adults (CARDIA) Study. JAMA Cardiol.

4. Connors LH, Lim A, Prokaeva T, Roskens VA, & Costello CE (2003) Tabulation of human transthyretin (TTR) variants, 2003. Amyloid 10(3): 160-184.

5. PipbergerHV (1978) Potentialities, problems and perspectives of ECG data processing. Verh Dtsch Ges Kreislaufforsch 44:33-37.

6. Bulawa CE, et al. (2012) Tafamidis, a potent and selective transthyretin kinetic stabilizer that inhibits the amyloid cascade. Proc Natl Acad Sci USA 109(24):9629-9634.

7. Judge DP, etal. (2019) Transthyretin Stabilization by AG10 in Symptomatic Transthyretin Amyloid Cardiomyopathy. J Am Coll Cardiol 74(3):285-295.

8. Lim KH, et al. (2016) Structural Changes Associated with Transthyretin Misfolding and Amyloid Formation Revealed by Solution and Solid-State NMR. Biochemistry 55(13): 1941-1944.

9. Lim KH, Dyson HJ, Kelly JW, & Wright PE (2013) Localized structural fluctuations promote amyloidogenic conformations in transthyretin. J Mol Biol 425(6):977-988.

10. Terry CJ, et al. (1993) Structure of Met30 variant of transthyretin and its amyloidogenic implications. EMBO J 12(2):735-741.

11. Zhang Q & Kelly JW (2005) Cys-10 mixed disulfide modifications exacerbate transthyretin familial variant amyloidogenicity: a likely explanation for variable clinical expression of amyloidosis and the lack of pathology in C10S/V30M transgenic mice? Biochemistry 44(25):9079-9085.

12. Leach BI, Zhang X, Kelly JW, Dyson HJ, & Wright PE (2018) NMR Measurements Reveal the Structural Basis of Transthyretin Destabilization by Pathogenic Mutations. Biochemistry 57(30):4421-4430.

13. Riek R & Eisenberg DS (2016) The activities of amyloids from a structural perspective. Nature 539(7628):227-235.

14. Bergstrom J, et al. (2005) Amyloid deposits in transthyretin-derived amyloidosis: cleaved transthyretin is associated with distinct amyloid morphology. J Pathol 206(2):224-232.

15. Benson MD (2012) Pathogenesis of transthyretin amyloidosis. Amyloid 19 Suppl 1: 14-15.

16. Yazaki M, et al. (2000) Cardiac amyloid in patients with familial amyloid polyneuropathy consists of abundant wild-type transthyretin. Biochem Biophys Res Commun 274(3):702-706.

17. Pomfret EA, et al. (1998) Effect of orthotopic liver transplantation on the progression of familial amyloidotic polyneuropathy. Transplantation 65(7):918-925.

18. Stangou AJ, etal. (1998) Progressive cardiac amyloidosis following liver transplantation for familial amyloid polyneuropathy: implications for amyloid fibrillogenesis. Transplantation 66(2):229-233.

19. Liepnieks JJ & Benson MD (2007) Progression of cardiac amyloid deposition in hereditary transthyretin amyloidosis patients after liver transplantation. Amyloid 14(4):277-282.

20. Llado L, et al. (2010) Risk of transmission of systemic transthyretin amyloidosis after domino liver transplantation. Liver Transpl 16(12): 1386-1392.

21. Stangou AJ, Heaton ND, & Hawkins PN (2005) Transmission of systemic transthyretin amyloidosis by means of domino liver transplantation. N Engl JMed 352(22):2356.

22. Saelices L, et al. (2018) Amyloid seeding oftransthyretin by ex vivo cardiac fibrils and its inhibition. Proc Natl Acad Sci USA 115(29):E6741-E6750.

23. Westermark GT, Fandrich M, Lundmark K, & Westermark P (2018) Noncerebral Amyloidoses: Aspects on Seeding, Cross-Seeding, and Transmission. Cold Spring Harb PerspectMed 8(1).

24. Dasari AKR, et al. (2019) Transthyretin Aggregation Pathway toward the Formation of Distinct Cytotoxic Oligomers. Sci Rep 9(1):33.

25. Breydo L & Uversky VN (2015) Structural, morphological, and functional diversity of amyloid oligomers. FEBSLett 589(19 PtA):2640-2648.

26. Fandrich M (2012) Oligomeric intermediates in amyloid formation: structure determination and mechanisms of toxicity. J Mol Biol 421(4-5):427-440.

27. Knowles TP, Vendruscolo M, & Dobson CM (2014) The amyloid state and its association with protein misfolding diseases. Nat Rev Mol Cell Biol 15(6):384-396.

28. Reixach N, Deechongkit S, Jiang X, Kelly JW, & Buxbaum JN (2004) Tissue damage in the amyloidoses: Transthyretin monomers and nonnative oligomers are the major cytotoxic species in tissue culture. Proc Natl Acad Sci USA 101(9):2817-2822.

29. Kayed R, et al. (2003) Common structure of soluble amyloid oligomers implies common mechanism of pathogenesis. Science 300(5618):486-489.

30. Holmes BB, et al. (2014) Proteopathic tau seeding predicts tauopathy in vivo. Proc Natl Acad Sci USA 111(41):E4376-4385.

31. Meyer-Luehmann M, et al. (2006) Exogenous induction of cerebral beta-amyloidogenesis is governed by agent and host. Science 313(5794):1781-1784.

32. Marin-Argany M, et al. (2016) Cell Damage in Light Chain Amyloidosis: FIBRIL INTERNALIZATION, TOXICITY AND CELL-MEDIATED SEEDING. J Biol Chem 291(38):19813-19825.

33. Quintas A, Vaz DC, Cardoso I, Saraiva MJ, & Brito RM (2001) Tetramer dissociation and monomer partial unfolding precedes protofibril formation in amyloidogenic transthyretin variants. J Biol Chem 276(29): 27207-27213.

34. Pires RH, Karsai A, Saraiva MJ, Damas AM, & Kellermayer MS (2012) Distinct annular oligomers captured along the assembly and disassembly pathways of transthyretin amyloid protofibrils. PLoS One 7(9):e44992.

35. Faria TQ, et al. (2015) A look into amyloid formation by transthyretin: aggregation pathway and a novel kinetic model. Phys Chem Chem Phys 17(11):7255-7263.

36. Frangolho A, Correia BE, Vaz DC, Almeida ZL, & Brito RMM (2020) Oligomerization Profile of Human Transthyretin Variants with Distinct Amyloidogenicity. Molecules 25(23).

37. Hammarstrom P, Jiang X, Hurshman AR, Powers ET, & Kelly JW (2002) Sequence-dependent denaturation energetics: A major determinant in amyloid disease diversity. Proc Natl Acad Sci US A 99 Suppl 4:16427-16432.

38. Lashuel HA, Wurth C, Woo L, & Kelly JW (1999) The most pathogenic transthyretin variant, L55P, forms amyloid fibrils under acidic conditions and protofilaments under physiological conditions. Biochemistry 38(41):13560-13573.

39. Lundberg E, Olofsson A, Westermark GT, & Sauer-Eriksson AE (2009) Stability and fibril formation properties of human and fish transthyretin, and of the Escherichia coli transthyretin-related protein. FEBSJ276(7):1999-2011.

40. Zhao L, Buxbaum JN, & Reixach N (2013) Age-related oxidative modifications of transthyretin modulate its amyloidogenicity. Biochemistry 52(11):1913-1926.

41. Altland K & Winter P (2003) Polyacrylamide gel electrophoresis followed by sodium dodecyl sulfate gradient polyacrylamide gel electrophoresis for the study of the dimer to monomer transition of human transthyretin. Electrophoresis 24(14):2265-2271.

42. Thylen C, et al. (1993) Modifications of transthyretin in amyloid fibrils: analysis of amyloid from homozygous and heterozygous individuals with the Met30 mutation. EMBO J12(2):743-748.

43. Teng MH, et al. (2001) Amyloid and nonfibrillar deposits in mice transgenic for wild-type human transthyretin: a possible model for senile systemic amyloidosis. Lab Invest 81(3):385-396.

44. Li X, et al. (2018) Amyloid deposition in a mouse model humanized at the transthyretin and retinol-binding protein 4 loci. Lab Invest 98(4):512-524.

45. Westermark P, Sletten K, Johansson B, & Cornwell GG, 3rd (1990) Fibril in senile systemic amyloidosis is derived from normal transthyretin. Proc Natl Acad Sci USA 87(7):2843-2845.

46. Saelices L, et al. (2015) Uncovering the Mechanism of Aggregation of Human Transthyretin. JBiol Chem 290(48):28932-28943.

47. Jiang X, et al. (2001) An engineered transthyretin monomer that is nonamyloidogenic, unless it is partially denatured. Biochemistry 40(38):11442-11452.

48. Lai Z, Colon W, & Kelly JW (1996) The acid-mediated denaturation pathway of transthyretin yields a conformational intermediate that can self-assemble into amyloid. Biochemistry 35(20):6470-6482.

49. Sekijima Y, et al. (2005) The biological and chemical basis for tissue-selective amyloid disease. Cell. 121(1):73-85.

50. Sato T, et al. (2007) Endoplasmic reticulum quality control regulates the fate of transthyretin variants in the cell. EMBO J26(10):2501-2512.

51. Susuki S, et al. (2009) The Endoplasmic Reticulum-associated Degradation of Transthyretin Variants Is Negatively Regulated by BiP in Mammalian Cells. J Biol Chem 284(13):8312-8321.

52. Fairhead M & Howarth M (2015) Site-specific biotinylation of purified proteins using BirA. Methods Mol Biol 1266:171-184.

53. Hurshman AR, White JT, Powers ET, & Kelly JW (2004) Transthyretin aggregation under partially denaturing conditions is a downhill polymerization. Biochemistry 43(23):7365-7381.

54. Yokoyama T, Hanawa Y, Obita T, & Mizuguchi M (2017) Stability and crystal structures of His88 mutant human transthyretins. FEBSLett 591(13):1862-1871.

### Sequence Listing

1. GLNDIFEAQKIEWHE GPTGTGESKC PLMVKVLDAV RGSPAINVAM
HVFRKAADDT WEPFASGKTS ESGELHGLTT EEEFVEGIYK VEIDTKSYWK
ALGISPFHEH AEVVFTANDS GPRRYTIAAL LSPYSYSTTA VVTNPKE
Avitag-TTRV30M

2. MASHRLLLLC LAGLVFVSEA GPTGTGESKC PLMVKVLDAV
RGSPAINVAV HVFRKAADDT WEPFASGKTS ESGELHGLTT EEEFVEGIYK
VEIDTKSYWK ALGISPFHEH AEVVFTANDS GPRRYTIAAL LSPYSYSTTA VVTNPKE
Human TTR precursor

3. GPTGTGESKC PLMVKVLDAV RGSPAINVAV HVFRKAADDT
WEPFASGKTS ESGELHGLTT EEEFVEGIYK VEIDTKSYWK ALGISPFHEH
AEVVFTANDS GPRRYTIAAL LSPYSYSTTA VVTNPKE
Human TTR mature

4. GPTGTGESKC PLMVKVLDAV RGSPAINVAM HVFRKAADDT
WEPFASGKTS ESGELHGLTT EEEFVEGIYK VEIDTKSYWK ALGISPFHEH
AEVVFTANDS GPRRYTIAAL LSPYSYSTTA VVTNPKE
TTRV30M

5. GLNDIFEAQKIEWHE
Avitag

1. A method for detecting transthyretin (TTR) monomers in a biological sample from a subject comprising:
(a) contacting the sample with a TTR binding reagent for a sufficient time for the TTR binding reagent to bind to TTR monomers in the sample and form a complex comprising the TTR binding reagent and TTR monomers, the TTR binding reagent comprising:
(i) a variant TTR comprising a valine to methionine substitution at position 30 (TTRV30M); and
(ii) an affinity moiety molecule linked to the variant TTR; and
(b) detecting the presence of the complex.

2. The method of claim 1, wherein the affinity moiety is biotin.

3. The method of claim 1, wherein the affinity moiety is linked to the N-terminus of the TTR binding reagent.

4. The method of claim 1, wherein the TTR binding reagent has the sequence of, or is 90% similar to that of SEQ ID NO: 1.

5. The method of claim 1, wherein the method further comprises binding the complex to streptavidin.

6. The method of claim 1, wherein the TTR binding reagent is linked to a solid support.

7. The method of claim 6, wherein the solid support comprises a microplate or a bead.

8. The method of claim 1, wherein detecting comprises contacting the complexes with a detection reagent.

9. The method of claim 8, wherein the detection reagent is selected from the group comprising an antibody, an aptamer, a fluorescent reagent, a chemiluminescent reagent, and a colorimetric reagent.

10. The method of claim 9, wherein the antibody or aptamer is linked to a detectable marker comprising a fluorescent molecule, a luminescent molecule, a radioisotope, and an enzyme capable of catalyzing a detectable chemical reaction.

11. A kit comprising:
a TTR binding reagent comprising:
(i) a variant TTR comprising a valine to methionine substitution at position 30 (TTRV30M); and
(ii) an affinity moiety molecule linked to the variant TTR.

12. The kit of claim 11, wherein the affinity moiety is linked to the N-terminus of the TTR binding reagent.

13. The kit of claim 11, wherein the TTR binding reagent has the sequence of, or is at least 90% similar to that of SEQ ID NO: 1.

14. The kit of claim 11, wherein the TTR binding reagent is linked to a solid support.

15. The kit of claim 14, wherein the solid support is a microplate.

16. The kit of claim 14, wherein the solid support is a lateral flow device.

17. The kit of claim 11, wherein the affinity moiety is biotin.

18. The kit of claim 11, wherein the detection reagent is selected from the group comprising an antibody, an aptamer, a fluorescent reagent, a chemiluminescent reagent, and a colorimetric reagent.

19. The kit of claim 18, wherein the antibody or aptamer is linked to a detectable marker comprising a fluorescent molecule, a luminescent molecule, a radioisotope, and an enzyme capable of catalyzing a detectable chemical reaction.

20. The kit of claim 11, wherein the TTR binding reagent comprises at least one polypeptide consisting of SEQ ID NO: 1.
